(19) United States
(12) Patent Application Publication
(10) Pub. No.: US 2025/0338869 A1
(43) Pub. Date: Nov. 6, 2025

(54) PREPARATION OF FUNCTIONAL PROTEINS OF A MICROORGANISM WITH REDUCED LIPID AND/OR NUCLEIC ACID CONTENT

(71) Applicant: ProteinDistillery GmbH, Ostfildern (DE)

(72) Inventors: Michael BAUNACH, Esslingen (DE); Christoph PITTER, Esslingen (DE); Tomas KURZ, Kiel (DE)

(21) Appl. No.: 18/867,088

(22) PCT Filed: May 24, 2023
(86) PCT No.: PCT/EP2023/063965

§ 371 Date: Nov. 19, 2024

(30) Foreign Application Priority Data
May 25, 2022 (EP) 22175426.0

(51) Int. Cl. A23J 1/18; A21D 2/26; A23C 11/10; A23J 3/20; A23J 3/22; A23L 15/00; A23L 29/269; B01D 15/36; B01D 17/02; B01D 61/14; B01D 69/02; C12N 1/06

(52) U.S. Cl. CPC A23J 1/18; A21D 2/267; A23C 11/10; A23J 3/20; A23J 3/22; A23J 3/227; A23L 15/35; A23L 29/269; B01D 15/363; B01D 17/0217; B01D 61/145; B01D 69/02; C12N 1/06; B01D 2315/16; B01D 2325/20

(57) ABSTRACT

The present invention relates to a method of preparing native protein of a microorganism comprising lysing the microorganism, separating lipids from the supernatant, filtering the supernatant and optionally separating nucleic acids, sterilizing the supernatant and/or drying the supernatant. The native proteins can be used for preparing protein preparations which are applied in the production of food or dietary supplements. The invention also relates to food products or dietary supplements comprising the protein preparations.

### FIELD OF THE INVENTION

The present invention relates to a method of preparing native protein of a microorganism with reduced lipid content and/or nucleic acid content. The resulting protein preparations can be used for the production of food or dietary supplements.

### BACKGROUND OF THE INVENTION

Protein-rich foods derived from microorganisms such as fungi, bacteria or algae are summarized under the term Single Cell Proteins (SCP). Compared to plant or animal protein sources, they offer the advantage that neither large cultivation areas nor high water consumption is required, and production is not dependent on seasonal or geographical factors. The high growth rates of microorganisms also make it possible to produce a large amount of microbial protein in a short time. The use of microorganisms as food has existed for some time, but the single-cell protein (SCP) products available on the market today consist largely of the untreated microorganisms itself.

Despite the high protein content of microorganisms, mainly non-functional protein concentrates from microorganisms are currently available. This is due to the fact that for preparation of the protein, procedures are used in which the proteins are irreversibly damaged and lose their physiological properties. As a result, the proteins lose functional properties such as solubility and texture properties, making them unattractive for use in the production of alternative food systems. Protein functionality is decisive for the physiochemical properties of protein in food systems and influences the behavior during preparation, processing, storage, and consumption and contributes to sensory as well as textual properties of food systems. The denaturation of proteins leads to a reduction of the functional properties and the resulting proteins are therefore no longer usable in many areas of the food industry.

Another aspect is that the presence of lipids impairs shelf life of the food products as they become rancid and cause an unpleasant taste. In addition, microorganisms usually have a high concentration of nucleic acids. Similar to the consumption of purine-rich foods such as meat, sausage and offal, excessive consumption can lead to increased uric acid levels which may cause pathological effects such as arthritis (gout), tophi or urinary calculus.

According to known methods, the extraction of lipids from microbial cell lysates is usually carried out with organic solvents (e.g., hexane, methanol and tetrahydrofuran). Extraction is laborious and associated with enormous costs, as the solvents used are toxic, volatile and highly flammable and thus pose a high health and fire risk. Further, the removal of the solvent residues is done by distillation at elevated temperature, which leads to the denaturation of proteins and thus affects their functional properties. The effort required for analysis of residual solvent should also not be underestimated, as solvents are only permitted in small residual quantities in the food products. For, example, U.S. Pat. No. 4,206,243 describes extraction of lipids from a microbial cell mass with ammonia or ammonium hydroxide and isopropanol or an organic solvent such as an alcohol. DE 2 328 628 describes a process of obtaining microbial protein wherein lipoid components are extracted with alcohol.

Supercritical fluid extraction (SFE supercritical CO₂ extraction) offers an alternative to the conventional extraction of fatty acids. However, this still needs to be investigated for its commercial feasibility on an industrial scale for the extraction of lipids from microbial cell lysates.

The conventional methods for reducing the nucleic acid content can be divided into chemical, enzymatic and ion exchange methods.

The chemical methods involve increased temperature or increased/decreased pH, which leads to the denaturation of the proteins and thus impairs the functional properties of the proteins. Furthermore, the nutritional safety of the isolated proteins is compromised due to the formation of potentially toxic compounds such as lysinoalanine. Another chemical method for depleting nucleic acids is precipitation with polymers such as polyethylenimine, but this method leads to a high protein loss of about 30%.

Enzymatic treatment by activating endogenous ribonucleases at elevated temperature requires a still active microorganisms and leads to protein denaturation. In addition, the protein loss with this method is approx. 33-35%.

Further, GB 2 101 606 describes column chromatography with anion exchange for removal of nucleic acids from homogenates of microorganisms. However, column chromatography of cell homogenates is limited due to blocking of packed columns by the unpurified viscous samples and the associated disturbances due to a reduced flow rate during the process. Moreover, a protein loss of 30-45% must be expected.

The above-mentioned methods for the reduction of nucleic acids generally have high production costs because the chemicals used cannot be recycled or can only be recycled uneconomically or large quantities of anion exchange material are required for the column and there is a high loss of proteins. This affects the economic practicability of such processes.

Further, WO 2020/127951 describes a method of preparing a functional protein concentrate. However, the method does not relate to separation of lipids and/or nucleic acids. Similarly, US 2022/071231 A1 and WO 2022/05287 A1 describe methods for preparing protein preparations from S. cerevisiae and Baker's yeast without referring to a step of lipid reduction and/or nucleic acids reduction.

Therefore, there is a need for a method for preparing microbial protein from a microorganism with a reduced lipid content and/or reduced nucleic acid content while retaining functional properties of the proteins.

It is an object of the invention to provide a method of preparing native microbial proteins with high yield and reduced lipid content thereby improving the shelf-life and flavor of the protein preparation.

It is a further object of the invention to provide a method of preparing native microbial proteins with improved functional properties for the production of food or dietary supplements, particularly in the field of vegan food production.

It is a further object of the invention to provide a method of preparing native microbial proteins with reduced nucleic acid content thereby increasing the quality for human consumption.

The objects of the invention are achieved by the subject matter of the independent claims. Preferred embodiments are subject of the dependent claims.

### SUMMARY OF THE INVENTION

The present invention relates to a method of preparing native protein of a microorganism comprising:
a) providing the microorganism, and optionally subjecting the microorganism to one or more pre-treatment step(s),
b) lysing the microorganism thereby obtaining a lysate comprising an aqueous liquid fraction comprising lipid and solved native protein of the microorganism, further comprising a step b1) of clearing the lysate, preferably by centrifugation or filtration,
c) separating the lipid from the aqueous liquid fraction using mechanical means thereby obtaining an aqueous liquid fraction wherein the aqueous liquid fraction is a lipid reduced aqueous liquid fraction comprising the solved native protein of the microorganism, wherein separating the lipid is performed by a centrifugal three-phase separator,
d) filtrating the aqueous liquid fraction thereby obtaining a solution comprising purified solved native protein of the microorganism and an aqueous solvent, preferably water or a saline solution,
e) optionally removing at least a part of the aqueous solvent,
f) optionally sterilizing the solution thereby obtaining a sterilized solution comprising purified solved native protein of the microorganism and an aqueous solvent, and
g) optionally removing at least a part of the aqueous solvent of the sterilized solution.

The present invention further relates to a protein preparation obtainable by the method according to the invention.

The present invention further relates to a protein preparation derived from a microorganism, preferably a single cell microorganism, comprising a gel forming capacity with about 1% to 10% of the protein preparation per total weight of a solution consisting of the protein preparation and water after heat treatment, preferably without syneresis, and optionally:
a) at least about 70% (w/w), preferably at least about 75% (w/w), more preferably at least about 85% (w/w) and most preferably at least about 95% (w/w) of protein by dry weight of the protein preparation,
b) about 110 mg/g or less, preferably about 50 mg/g or less, more preferably about 40 mg/g or less, most preferably about 20 mg/g or less of lipid by dry weight of the protein preparation,
c) a water binding capacity of about 4 g/g or more, preferably about 5 g/g or more, more preferably about 6.5 g/g or more by dry weight of the protein preparation after heat treatment,
d) a gel forming capacity with about 2%, about 3%, about 5%, or about 5.5% of the protein preparation per total weight of a solution consisting of the protein preparation and water after heat treatment, preferably without syneresis, and/or
e) about 10% (w/w) or less, more preferably about 5.5% (w/w) or less and more preferably about 2.5% (w/w) nucleic acid by dry weight of the protein preparation.

The present invention further relates to a method for preparing a protein gel comprising:
(a) providing a protein preparation according to any one of claims 9 to 11,
(b) mixing the protein preparation with an aqueous carrier fluid, and
(c) heating the mixture to a temperature of at least about 55° C. to provide the protein gel.

The present invention also relates the use of the protein preparation of the invention for preparing a food product, preferably for human or animal use, or a dietary supplement

Further, the invention relates to a dietary supplement or a food product comprising the protein preparation of the invention.

Further, the invention relates to a method of obtaining native protein of a microorganism comprising:
a) providing a microorganism, and optionally subjecting the microorganism to one or more pre-treatment step(s),
b) lysing the microorganism thereby preparing a lysate comprising an aqueous liquid fraction comprising nucleic acid and solved native protein of the microorganism, further comprising a step b1) of clearing the lysate, preferably by centrifugation or filtration,
c) separating the nucleic acid from the aqueous liquid fraction comprising anion exchange chromatography and/or anion mixed-mode chromatography comprising:
   i) adding a nucleic acid adsorbent immobilized to a solid support, preferably to a free-floating solid support to the aqueous liquid fraction,
   ii) optionally stirring or shaking, and
   iii) separating the nucleic acid bound to the nucleic acid adsorbent immobilized to the solid support, preferably by sedimentation and optionally filtration,
   thereby obtaining an aqueous liquid fraction wherein the aqueous liquid fraction is a nucleic acid reduced aqueous liquid fraction,
d) filtrating the aqueous liquid fraction thereby obtaining a solution comprising purified solved native protein of the microorganism and an aqueous solvent, preferably water or a saline solution,
e) optionally removing at least a part of the aqueous solvent,
f) optionally sterilizing the solution thereby obtaining a sterilized solution comprising purified solved native protein of the microorganism and an aqueous solvent, and
g) optionally removing at least a part of the aqueous solvent of the sterilized solution of step f),
   wherein preferably the method further comprises a step of separating lipid from the aqueous liquid fraction.

### DETAILED DESCRIPTION OF THE INVENTION

Unless otherwise defined, all technical and scientific terms used herein have the same meaning as commonly understood by the skilled person in the art to which this disclosure belongs.

It is noteworthy that the use of the undefined article “a” or “an” means one or more unless it is stated otherwise. Also, the term “about” as used herein means+/−10% if not stated otherwise.

The term “comprising” or “comprises” as used herein means “including, but not limited to”. The term is intended to be open-ended, to specify the presence of any stated features, elements, integers, steps, or components, but not to preclude the presence of addition of one or more other features, elements, integers, steps, components, or groups thereof. The term “comprising” or “comprises” thus includes the more restrictive terms “consisting of” and “consisting essentially of”. In one embodiment, the term “comprising” or comprises” as used throughout the application and in particular within the claims may be replaced by the term “consisting of”.

The present invention relates to a method of producing native protein from a microorganism.

The inventors could show that a mild processing method yields microbial proteins which are optimally suited for use in food products. In particular, the method involves safe and cost-effective steps of separating lipids and/or nucleic acids while retaining the functional properties, improving the tase, the shelf-life and health aspects of the microbial protein preparations thereby allowing a versatile use of the protein preparations in the production of food and dietary products. Specifically, the proteins of the invention have increased water binding properties, powder solubility, emulsion and foaming properties compared to conventional plant proteins and a gel forming capacity comparable to egg white which makes them particularly suitable as substitute or equivalent in vegan, i.e., non-animal food.

In one aspect, the invention provides a method of preparing native, i.e., not denatured, protein of a microorganism comprising:
a) providing the microorganism, and optionally subjecting the microorganism to one or more pre-treatment step(s),
b) lysing the microorganism thereby obtaining a lysate comprising an aqueous liquid fraction comprising lipid and solved native protein of the microorganism, further comprising a step b1) of clearing the lysate, preferably by centrifugation or filtration,
c) separating the lipid from the aqueous liquid fraction using mechanical means thereby obtaining an aqueous liquid fraction wherein the aqueous liquid fraction is a lipid reduced aqueous liquid fraction comprising the solved native protein of the microorganism, wherein separating the lipid is performed by a centrifugal three-phase separator,
d) filtrating the aqueous liquid fraction thereby obtaining a solution comprising purified solved native protein of the microorganism and an aqueous solvent, preferably water or a saline solution,
e) optionally removing at least a part of the aqueous solvent,
f) optionally sterilizing the solution thereby obtaining a sterilized solution comprising purified solved native protein of the microorganism and an aqueous solvent, and
g) optionally removing at least a part of the aqueous solvent of the sterilized solution. The person skilled in the art will understand that by performing steps a), b), c) and d) and optional steps e), f) and g) of said method, the native protein (such as a native protein preparation) is obtained. The method according to the present invention does not contain a step of blending previously separated fractions.

The term “protein of a microorganism” refers to a protein which is present in a microorganism. It includes but is not limited to a specific type of protein, such as metabolic, transport, storage or structural proteins. Further, a protein of a microorganism may refer to endogenous proteins of the microorganisms. A protein of a microorganism may also refer to recombinantly expressed proteins of the microorganism, e.g., proteins which increase the value of the protein preparation in food production. In one embodiment, the protein of the microorganism is an endogenous protein of the microorganism.

A native protein of the invention is a protein which retains its functional properties. In one embodiment, the protein retains its natural physical properties such as solubility, water binding, oil binding, emulsion or foaming properties. In one embodiment, the protein retains its natural structural properties.

The type of microorganism that is used in the present invention is not specially limited. In one embodiment, the microorganism is a eukaryotic microorganism. In a further embodiment, the microorganism is a eukaryotic microorganism selected from the group consisting of a fungus, a yeast, and an alga.

In one embodiment, the microorganism is a fungus, preferably a fungus selected from the group consisting of Aspergillus spp., preferably Aspergillus flavus; Aspergillus niger; Aspergillus ochraceus or Aspergillus oryzae; Rhizopus chinensis; Trichoderma harzianum; Cladosporium cladosporioides and Chrysonilia sitophilia. In a more preferred embodiment, the fungus is Aspergillus niger.

In another embodiment, the microorganism is an alga, preferably an alga selected from the group consisting of Aphanizomenon flos-aquae; Aphanothece microscopica; Arthrospira spp., preferably Arthrospira maxima (Spirulina maxima) or Arthospira platensis (Spirulina platensis); Chlorella spp., preferably Chlorella vulgaris; Chlorella pyrenoidosa or Chlorella sorokiana; Euglena gracilis; and Scenesdesmus obliquus. In a more preferred embodiment, the alga is selected from the group consisting of Arthrospira maxima (Spirulina maxima), Arthospira platensis (Spirulina platensis), Chlorella vulgaris and Euglena gracilis.

In a preferred embodiment, the microorganism is a yeast. In one embodiment, the yeast is an alcohol-producing yeast. In one embodiment, the yeast is selected from the group consisting of Saccharomyces spp., preferably Saccharomyces pastorianus, Saccharomyces cerevisiae, Saccharomyces carlsbergensis, Saccharomyces bayanus, Saccharomyces ellipsoides, Saccharomyces uvarum, or Saccharomycodes ludwigii; Pichia spp., preferably Pichia pastoris or Pichia anomala (Wickerhamomyces anomalus); Debaryomyces hansenii; Schizosaccharomyces spp., preferably Schizosaccharomyces pombe; Hansenula spp.; Schwanniomyces occidentalis; Zygosaccharomyces rouxii; Amoco Torula; Torulaspora delbruecki; Saccharomycopsis fibuligera; Debaryomyces hansenii; Brettanomyces bruxellensis; Candida spp., preferably Candida intermedia, Candida arborea, Candida guilliermondii, Candida halophila, Candida krusei, Candida langeronii, Candida lipolytica, Candida parapsilosis, Candida pararugosa, Candida tropicalis, Candida novellas, or Candida utilis; Rhodotorula glutinis; Cyberlindnera jadinii; Hanseniaspora uvarum; Kluyveromyces fragilis; Kluyveromyces marxianus; Lipomyces spp.; Torulopsis spp.; and Yarrowia lipolytica.

In a particularly preferred embodiment, the yeast is selected from the group consisting of Saccharomyces spp., preferably S. cerevisiae, S. carlsbergensis, S. bayanus, S. ellipsoides, S. uvarum, S. ludwigii or S. pastorianus, Pichia spp., preferably P. pastoris; Hansenula spp.; Candida spp., preferably C. utilis; Torulopsis spp.; and Yarrowia lipolytica.

In a most preferred embodiment, the microorganism is a yeast of Saccharomyces spp., more preferably from S. cerevisiae, S. pastorianus or S. carlsbergensis.

In another embodiment the microorganism is a prokaryotic microorganism.

In one embodiment, the microorganism is bacterium, preferably a bacterium selected from the group consisting of Bacillus spp., preferably Bacillus cereus, Bacillus licheniformis, Bacillus pumilis, Bacillus subtilis or Bacillus megaterium; Lactobacillus spp., preferably Lactobacillus casei, Lactobacillus salivarius, Lactobacillus bulgaricus, Lactobacillus delbrueckii, Lactobacillus helveticus, Lactobacillus pentisus, Lactobacillus plantarum, Lactobacillus curvatus or Lactobacillus sake; Pedioccoccus spp., preferably Pedioccoccus acidilactici or Pedioccoccus pentosaceus; Lactococcus spp., preferably Lactococcus lactis; Leuconostoc mesenteroides; Oenococcus oeni; Pseidomonas fluorescens; Corynebacterium spp., preferably Corynebacterium ammoniagenes or Corynebacterium glutamicum; Cupriavidus necator; Methylomonas spp.; Rhizospheric diazotrophs; Rhodopseudomonas palustris; Aeromonas hydrophila; Methylococcus capsulatus; Ralstonia spp.; Brevibacillus agri; Aneurunibacillus spp.; Acromobacter Calcoacenticus; Spirulina ssp., preferably Spirulina maxima or Spirulina platensis; Xanthomonas spp., preferably Xanthomonas campestris; Acetobacter spp.; Gluconobacter spp.; Staphylococcus spp., preferably Staphylococcus carnosus or Staphylococcus xylosus; and Ideonella sakalensis. In a preferred embodiment, the bacterium is selected from the group consisting of Bacillus subtilis, Lactobacillus spp., Corynebacterium glutamicum, Methylomonas spp., and Xanthomonas spp.

In one embodiment, the microorganism is a single cell organism.

In the first step of the above method, the microorganism is provided in any form including but not limited to a microorganism in suspension. In a simple embodiment, the suspension of the microorganism is a cell-containing medium that was used for culturing of the microorganism, e.g., waste product of a beer brewing process, preferably spent yeast. The cell-containing medium can be used directly in the sense of step b). Alternatively, the cell-containing medium can be subjected to one or more pre-treatments steps comprising filtration, sieving, washing, and/or centrifugation. For example, the microorganism can be harvested from the culture medium by centrifugation. The centrifugation step may be preceded by a step of filtration to remove cell medium constituents. Subsequently, the harvested microorganism may be subjected to one or more washing steps to remove residual cell medium constituents, optionally followed by resuspension in a suitable buffer or water. Preferably, the pre-treatment step(s), particularly the washing step, is/are performed at a temperature which does not exceed 45° C., preferably at a temperature of about 30° C. to 40° C., preferably at about 37° C.

In one embodiment the suspension is preferably an aqueous suspension. In principle, the method of the invention can be carried out in any volume, from lab scale, e.g., about 1-10 liters, to industrial scale. In one embodiment, the suspension has a volume of about 1 liter or more, about 4 liters or more, about 5 liters or more, about 10 liters or more, about 20 liters or more, about 50 liters or more, about 100 liters or more, about 200 liters or more, about 300 liters or more, about 400 liters or more, about 500 liters or more, about 600 liters or more, about 700 liters or more, about 800 liters or more, about 900 liters or more, about 1 000 liters or more, about 5 000 liters or more, about 10 000 liters or more. In a further embodiment, the suspension is adjusted to a dry matter content of about 5%-20%, preferably about 10% to 15%, more preferably about 12% to 14% per weight percent of the total weight of the suspension. The dry matter content can be determined by any method known in the art including a commercially available halogen moisture analyzer, e.g., with the MB 35 Halogen OHAUS Europe GmbH (105° C.±2° C.). After determining the dry matter content, the suspension can be diluted or concentrated to achieve the above range.

In a particular preferred embodiment, the suspension of the microorganism is a waste product of a beer brewing process, preferably spent yeast. In one embodiment the pre-treatment step comprises filtration or sieving of the suspension. Filtration or sieving may by useful to remove residual hop. Filtration or sieving can be carried out with a sieve, e.g., a nylon sieve or a vibration sieve, preferably a stainless-steel vibration sieve. Filtration can also be carried out with a filter bag. The mesh size for filtration or sieving is from about 110 μm to about 140 μm, preferably about 120 μm to about 130 μm, more preferably about 125 μm. The pre-treatment step may further comprise a step of centrifugating the suspension to remove spent yeast. Centrifugation is advantageously carried out at 2 000 g to 4 000 g, preferably at 3 000 g. The pre-treatment step may further comprise contacting, and preferably incubating, the microorganism with a polysorbate solution, e.g., Tween 80, more preferably an alkaline polysorbate solution. This step may be useful to remove the spent yeast thereby improving the taste of the protein. Preferably, contacting is performed at a temperature of about 35° C. to about 40° C., preferably about 37° C. Subsequently, the microorganism is washed, preferably, the washing is repeated until the pH of the suspension reaches about pH 5.5 to 7.0, preferably pH 6.4.

Step b) of the inventive method comprises lysing the microorganism thereby obtaining a lysate comprising an aqueous liquid fraction comprising lipid and solved native protein of the microorganism. More precisely, the aqueous liquid fraction comprises a lipid fraction and an aqueous fraction comprising the solved native protein of the microorganism. The person skilled in the art would understand that the aqueous liquid fraction may further comprise nucleic acids. The aqueous liquid fraction may further comprise a solid fraction comprising, e.g., residual cell debris which was not removed by clearing the lysate. In one embodiment, the pH is set from about 6.3 to 8.5, preferably about 6.4 prior to lysis. It will be understood by those skilled in the art that the specific method for lysing will generally depend on the specific microorganism. For example, the microorganisms which are useful in the context of the present invention such as yeasts, fungi, algae or bacteria have a cell wall and a plasma membrane which both need to be disrupted for release of the proteins. Another factor to be considered is that the method of lysis must be chosen as to retain the native structure of the proteins of the microorganism. In this regard, in one embodiment of the method of the invention steps b) to g), preferably steps b) to d), particularly steps b) and d), are performed at a temperature of about 40° C. or less (up to a temperature of about 2° C. to 8° C.), preferably of about 30° C. or less, such as about 30° C. to 2° C., preferably about 30° C. to 8° C., more preferably about 30° C. to 20° C. Performing lysis in this temperature range avoids undesired protein degradation which would lead to a loss of the functional properties of the proteins and/or a decreased activity of proteases which are also released from the microorganism upon lysis. Accordingly, in preferred embodiment of the method of the invention lysis comprises mechanical lysis, such as high-pressure homogenization or bead milling; or physical lysis such as sonoporation and/or electroporation.

The beads can be made of steel, ceramic, rubber, or glass. For the purpose of the invention, the use of ceramic beads, e.g., zirconia/silicon carbide beads or glass beads have been turned out to be particularly useful. Further, the beads, e.g., the ceramic beads or glass beads may have a size of about 0.05 mm to 0.7 mm, preferably about 0.5 mm to 0.6 mm, more preferably about 0.5 mm. Further, the bead fill volume may range between about 40% to about 90%, preferably about 50% to 80%, more preferably about 60% to about 70%. In order to increase the efficacy of the milling, it is favorable to use an accelerator which accelerates the grinding media, i.e., the beads. It has further been found that an energy input of 0.01 to 0.2 kWh/kg slurry, e.g., a suspension of the microorganism, is useful.

The efficacy of the cell disruption can be monitored by microscopic control, e.g., phase contrast method; or the protein content in the supernatant after centrifugation, e.g., (Pierce™ BCA Protein Assay Kit, Thermo Scientific). These methods are known to the person skilled in the art. The protein content in the supernatant after 95% cell disruption is about 40 mg/ml to 80 mg/ml, preferably about 50 mg/ml to about 70 mg/ml, more preferably about 55 mg/ml to 60 mg/ml.

Prior to separating the lipids in step c), the disrupted cell suspension, i.e., the lysate is cleared. This step separates insoluble cell debris, e.g., chromosomal DNA or cell wall to obtain an aqueous liquid fraction comprising lipid and solved native protein of the microorganism. Thus, the method of the invention comprises a further step b1) of clearing the lysate. The clearing can be performed by different methods including but not limited to centrifugation or filtration. In one embodiment, clearing is performed by centrifugation at about 2 000 g to 25 000 g, at about 2 000 g to 20 000 g, preferably at about 5 000 g to 19 000 g, more preferably about 6 000 g to 17 000 g at about 17 000 g. Clearing may also be performed stepwise by centrifugation at about 2 000 g to about 7 000 g followed by further centrifugation of the supernatant at about 5 000 g to 19 000 g, preferably about 6 000 g to 17 000 g, more preferably at about 17 000 g. Such a clearing (centrifugation or filtration) step does not reduce the lipid content in the aqueous liquid fraction.

Step c) of the inventive method comprises separating the lipid from the aqueous liquid fraction thereby obtaining an aqueous liquid fraction wherein the aqueous liquid fraction is a lipid reduced aqueous liquid fraction comprising the solved native protein of the microorganism. Separating the lipid, including lipophilic substances, is advantageous because fat-soluble (lipophilic) substances have a strong influence on the flavor, in particular by causing a rancid taste. Microorganisms, especially yeasts (Saccharomyces spp.), have a high content of unsaturated fatty acids such as oleic acid, palmitoleic acid and linoleic acid. In addition, yeasts contain so-called lipid particles, primarily non-polar lipids and sterols, which serve as building blocks for membrane lipid synthesis. Fatty acid residues, especially of unsaturated fatty acids, are particularly susceptible to oxidation processes and therefore tend to become rancid very quickly, which has a negative effect on the shelf-life and taste of the food products or dietary supplements produced with the protein. The term “lipid” as used herein is a collective term that refers to biomolecules soluble in nonpolar solvents, such as hydrocarbons (e.g., hexane). It may also be referred to as lipids, lipid fraction or lipid-containing fraction. In living organisms, lipids are mainly used as structural components in cell membranes, as energy stores or as signal molecules. Most biological lipids are amphiphilic, i.e. they have a lipophilic hydrocarbon residue and a polar hydrophilic head group, which is why they form micelles or membranes in polar solvents such as water. The term fat is often used as a synonym for lipids, but fat represent only one subgroup of lipids (namely, the triglyceride group). Lipids also encompass molecules such as fatty acids and their derivatives (including tri-, di-, monoglycerides, and phospholipids), as well as other sterol-containing metabolites such as cholesterol. Lipids can be divided into seven groups: Fatty acids, triacylglycerides (fats and oils), waxes, phospholipids, sphingolipids, lipopolysaccharides, and isoprenoids (steroids, carotenoids, etc.). Non-natural or synthetic molecules are typically not referred to as lipids.

For the purpose of the invention the lipids are separated using mechanical means because conventional lipid separating methods with organic solvents involve toxic solvents which is inacceptable for providing a protein preparation for the production of food. Further, regulations for food production limit the amounts of organic solvents which requires removal of residual solvent by distillation. Distillation takes place at elevated temperature and leads to the denaturation of proteins and thus affects their functional properties. Moreover, mechanical methods are an effective approach as they are less dependent on the type of microorganism being processed and cause less contamination. More specifically mechanical means are a centrifugal separator that separates the lipid from the aqueous liquid fraction. This separation is based on a different density of the lipid and the lipid reduced aqueous liquid fraction comprising the solved native protein of the microorganism. Thus, the centrifugal separator is a centrifugal three-phase separator. According to the method of the invention the step of separating the lipid is therefore performed by a centrifugal (three-phase) separator, such as a skimming separator and a three-phase decanter. The term “mechanical means as used herein does not refer and excludes extraction with organic solvents. The inventors have surprisingly found a safe and cost-effective method for reducing the lipids from microbial cell lysates or aqueous liquid fractions thereof while retaining the functional properties of the proteins, which is based on different density between the lipid and the lipid reduced aqueous liquid fraction comprising the solved native protein of the microorganism.

The person skilled in the art would understand that for reduction of lipid content a centrifugal three-phase separator (e.g., skimming separation) is required, which is different to a separator with 2-phase separation (clarifier, sediment centrifuges). A separator with 2-phase separation, such as centrifugal clarifiers, can be used to separate suspensions (2-phase separation) consisting of a solid fraction (sludge) and a liquid fraction. The suspension is fed into the centrifuge and the centrifugal force separates the solid particles from the liquid. The solid particles settle on the bottom of the centrifuge due to gravity and the clear liquid is tapped from the top. In a separator with 3-phase separation, particularly using a skimming separator, fat/lipid-containing solutions (3-phase separation) consisting of a light liquid portion, a heavy liquid portion and a solids portion (sludge) can be separated. The fat/lipid-containing solution is placed in the bowl and the centrifugal force separates the two liquids as well as the solids by gravity. The two liquids can then be discharged from the drum through special channels. The solids remain in the drum or alternatively are discharged discontinuously.

The term “skimming separator” as used herein refers to a 3-phase disc stack separator. It contains a disk stack with a large number of disc plates arranged parallel to each other. The disc plates specifically have riser holes arranged in the centre of the disc plate. The liquid is introduced into the main separation zone through riser channels. From there the light liquid phase (fat/lipid solution) flows towards the axis of rotation, while the heavy liquid phase (defatted protein solution) moves towards the bowl wall. An additional impeller disc above the disc stack prevents the liquid phases from mixing after separation. The disc stack not only separates the light and heavy liquid phases, but also separates solid particles. The centrifugal force causes the solid particles to separate in the disc stack and slide down the underside of the discs into the solids compartment of the bowl. The disc stack introduces more settling area. This added surface area speeds up the separation process exponentially. By contrast a three-phase decanter uses a screw conveyor for separation. Decanters and disc stack separators (skimming separators) are both centrifugal separators. In the context of separating the lipid from the aqueous liquid fraction in the method of the present invention the centrifugal separator is a centrifugal three-phase separator (e.g., a three-phase decanter or a skimming separator). Centrifugation is a piece of separation technology that allows for high-speed separation of immiscible (non-mixable) liquids and particles by gravity, which is also applied by a centrifugal three-phase separator. The term “centrifugal separator” as used herein refers to a centrifugal three-phase separator and typically contains a rotating bowl (preferably comprising means such as disc stacks or a screw conveyor) and does not include and is distinct from a classical centrifuge, i.e., using a container (such as a tube or a bucket) comprising the sample or fluid to be separated placed in a rotor.

According to the invention, separating the lipid from the aqueous liquid fraction by mechanical means in step c) is performed by a centrifugal separator, specifically a centrifugal three-phase separator. A preferred centrifugal separator is a centrifuge which contains a disc stack designed with vertically arranged riser holes (i.e., a skimming separator). The lysate or the aqueous liquid fraction comprising lipid and solved native protein of the microorganism flows in through the vertical riser holes. Due to the different density and under the influence of the centrifugal force, the lipids of the lysate or the aqueous liquid fraction can thus be separated from the lysate or the aqueous liquid fraction comprising the solved native protein of the microorganism. Since the lipids have a lower density, they flow inwards in the direction of the axis of rotation. Thus, the lipids can be separated via an axially arranged outlet and the total lipids content can be reduced. The centrifugal (three-phase) separator can be orientated vertically or horizontally. In one embodiment, the feed rate is from about 0.5 L/min to 50 L/min, about 1 l/min to 20 L/min, about 5 L/min. to 10 L/min. In another embodiment the feed rate is from about 100 I/h to about 20 000 l/h, preferably from about 500 l/h to about 15 000 l/h, more preferably from about 1 000 l/h to about 10 000 l/h. Further, for the purpose of the present invention, it has been found that a temperature of 40° C. or less is favorable (up to a temperature of about 2° C. to 8° C.), particularly 30° C. or less, such as 30° C. to 2° C., preferably 30° C. to 8° C., preferably 30° C. to 20° C. is particularly favorable for separating lipids. In particularly preferred embodiment, separating the lipid is performed with a skimming separator or a three a three-phase decanter, e.g., Tricanter® (Flottweg). A three-phase decanter separates lipid and advantageously also a solid fraction, if present, from the aqueous liquid fraction comprising the solved native protein of the microorganism thereby improving the purity of the aqueous liquid fraction comprising the solved native protein of the microorganism.

The success of separating the lipids can be determined, e.g., by recording a UV spectrum (200-350 nm) after solvent extraction of the aqueous liquid fraction (the lipid reduced aqueous liquid fraction and/or the aqueous liquid fraction prior to lipid separation) or the lysate. This method is rapid and provide rapid results about the reduction of the unsaturated fatty acids and other lipophilic substances. A difference spectrum (UV absorption spectrum) between an untreated and a lipid reduced aqueous fraction or lysate is used. The success of the lipid reduction can then be compared and determined on the basis of the expression of the characteristic diene and triene fatty acid bands in the UV spectrum (200-350 nm). Thus, in one embodiment, the method of the invention further comprises quantifying lipid in the aqueous liquid fraction (e.g., the lipid reduced aqueous liquid fraction and/or the aqueous liquid fraction prior to separation) comprising:
i) contacting the aqueous liquid fraction with a lipophilic solvent, preferably hexane, thereby obtaining a lipophilic phase,
ii) measuring the absorption, preferably absorption in the UV wavelength range, of the lipophilic phase,
iii) comparing the absorption measured for the lipophilic phase with a reference absorption measured for the lipid, and
iv) quantifying the lipid in the lipophilic phase.

Clearing can also be performed after separation of the lipids. Thus, in one embodiment, the method of the invention comprises a further step c1) of clearing the aqueous liquid fraction which is a lipid reduced aqueous liquid fraction comprising the solved native protein of the microorganism.

In step d) of the inventive method, the aqueous liquid fraction is filtered to remove particles which are smaller in size than of 1 kDa to about 100 kDa, preferably of about 3 kDa to about 50 kDa, more preferably of about 5 kDa to about 30 kDa, most preferably of about 10 kDa. The particle removed by filtration are preferably smaller than 10 kDa, more preferably smaller than 5 kDa. Step d) comprises filtrating the aqueous liquid fraction, preferably the aqueous liquid fraction of step c), more preferably the lipid reduced aqueous liquid fraction comprising the solved native protein of the microorganism, thereby obtaining a solution comprising purified solved native protein of the microorganism and an aqueous solvent, preferably wherein the aqueous solvent is water or a saline solution. The steps of the method are performed in the order recited in the method of the invention.

In one embodiment filtration is step d) is performed via ultrafiltration. The separation principle of ultrafiltration is based on a membrane which allows passage of particles which are smaller in size than the pores of the membrane and of solvent while particle or molecules with a size larger than the pore size of the membrane are retained. Thus, the filtration step results in two fractions, the permeate (solvent with particles or molecules smaller than the pore size of the membrane) and the retentate (solvent with particles or molecules larger than the pore size of the membrane). Membranes, preferably hydrophilic membranes, used for ultrafiltration have a molecular weight cut-off in a range of about 1 kDa to about 100 kDa, preferably of about 3 kDa to about 50 kDa, more preferably of about 5 kDa to about 15 kDa, most preferably of about 10 kDa. For the purpose of the present invention, ultrafiltration is applied as tangential flow filtration. In one embodiment, the membrane is a hollow fiber membrane with a molecular weight cut-off of about 10-20 kDa.

In a further embodiment of step d) ultrafiltration is combined with diafiltration to change the liquid. During diafiltration, a solvent is continuously applied to the retentate in an ultrafiltration process until to the desired degree of exchange by the solvent is achieved. Thus, in the present invention diafiltration is applied to replace at least a part of the liquid of the aqueous liquid fraction by a solvent, preferably an aqueous solvent, more preferably water or a buffer, e.g., a saline solution. A saline solution is a mixture between a salt and water. In one embodiment, the salt is sodium chloride, ammonium sulfate, potassium phosphate, or ammonium chloride). In a preferred embodiment the saline solution is a sodium chloride solution preferably about 0.01% to about 5%, preferably about 0.5% to about 2%, more preferably about 0.9% to about 1.5% (w/v) sodium chloride solution. In one embodiment, the liquid of the aqueous liquid fraction is substantially, preferably completely, changed by the solvent. After diafiltration, the solution is preferably concentrated by a factor in the range of about 1.5 to about 4.5, preferably of about 2 to about 3.5, more preferably by a factor about 3. The factor is defined by total filtration starting volume/retentate volume. In one embodiment, the diavolume is within a range of about 0.0 to 5.0, preferably 0.3 to 5.0, more preferably 0.7. The diavolume is a relative volume and defined as product of the total volume introduced to the operation during diafiltration/retentate volume.

Optionally, the aqueous solvent of the solution obtained after filtration in step d) is removed at least a partially in step e) to further concentrate the solution, preferably at least about 50% of the solvent, preferably at least about 75%, more preferably at least about 90%, most preferably at least about 94% or even 98% of the solvent are removed. In one embodiment, the solution is dried to obtain a powder of the native protein of the microorganism. In essence, removing at least a part of the solvent in step e) and/or step g) may include any method including but not limited to spray-drying, vacuum-drying, drum-drying, fluidized bed drying or freeze-drying, preferably spray-drying. These methods are conventional methods and are known to the skilled person. In one embodiment, the product of step e) has a solvent content of about 4% to 40%, preferably about 4% to 30%, more preferably about 4% to 20% relative to the dry weight of the total product.

In yet a further embodiment of the method of the invention, the solution of step d) or step e), if present, may optionally be sterilized in step f).

For the purpose of the invention, it is important that sterilization does not degrade or denature the proteins which may result in a loss of functional properties of the protein. Suitable sterilization methods are known in the art and the skilled person will be aware of them, e.g., sterile filtration, ultra-high temperature processing (UHT), preferably for a part of a second, ultraviolet light (UV) processing, and Pulsed Electric Field (PEF) processing. In a preferred embodiment, sterilization is performed by sterile filtration. The principle is based on filtration with a membrane having pore sizes suitable for elimination of bacteria and fungus. Generally, any membrane filter system known in the art for sterile filtration can be used. These systems are known to the skilled person. Particularly useful in this regard are filter membranes with pore size of about 0.1 μm to 1 μm, preferably about 0.2 μm. For the purpose of the present invention, sterile filtration with a heterogenic membrane system, e.g., polyethersulfone (PES) double membrane (0.8 μm) and glass fiber membrane (0.2 μm) are found to be particularly useful.

Optionally, the aqueous solvent of the solution obtained after sterilizing in step f) is removed at least a partially to further concentrate the solution, preferably at least about 50% of the solvent, preferably at least about 75%, more preferably at least about 90%, most preferably at least about 94% or even 98% of the solvent are removed. In one embodiment, the solution is dried to obtain a powder of the native protein of the microorganism. In essence, removing at least a part of the solvent in step e) and/or step g) may include any method including but not limited to spray-drying, vacuum-drying, drum-drying, fluidized bed drying or freeze-drying, preferably spray-drying. These methods are conventional methods and are known to the skilled person. In one embodiment, the product of step g) has a solvent content of about 4% to 40%, preferably about 4% to 30%, more preferably about 4% to 20% relative to the dry weight of the total product.

In a specific embodiment, the method of the invention further comprises a step of separating nucleic acid, e.g., ribonucleic acids (RNA) or deoxyribonucleic acids (DNA). In one embodiment, separating nucleic acid is performed after step b) or b1), i.e., nucleic acids are separated from the lysate (or the cleared lysate). In another embodiment, separating nucleic acid is performed after step d), i.e., nucleic acids are separated from the solution of step d). In a preferred embodiment, separating nucleic acid is performed after step c), preferably from the lipid reduced aqueous liquid fraction comprising the solved native protein of the microorganism. Thus, the aqueous liquid fraction of step d) may be a lipid reduced aqueous liquid fraction or lipid reduced and nucleic acid reduced aqueous liquid fraction.

For the purpose of the present invention, separating nucleic acid can be performed at a temperature of about 40° C. or less, 30° C. or less, 20° C. or less, 10° C. or less, preferably about 30° C. or less and/or does not involve the use of low or high pH conditions to avoid denaturation of the protein and loss of the functional properties of the protein. Thus, in a preferred embodiment, separating nucleic acid comprises chromatography. Chromatography can be carried out in any volume, from lab scale to industrial scale. For example, the chromatography may be carried out in a volume of about 0.5 l to 2 l. The chromatography may also be carried out in a volume of about 1 l or more, 10 l or more, 20 l or more 50 l or more, 100 l or more, 200 l or more, 500 l or more, 1 000 l or more, 2 000 l or more 5 000 l or more, 7 000 l or more, or 10 000 l or more.

In principle, any type of chromatography is possible which is suitable for separation of nucleic acids. Those chromatography methods are known in the art and include anion exchange chromatography and/or anion exchange mixed-mode chromatography. According to the invention separating the nucleic acid from the aqueous liquid fraction comprises anion exchange chromatography and/or mixed-mode chromatography. In anion-exchange chromatography, a nucleic acid adsorbent is immobilized on a solid support, for example ceramic or resin, e.g., styrene-DVB. The nucleic acid adsorbent comprises a positively charged functional group, e.g., a quaternary ammonium compound. Nucleic acids with a negatively charged backbone can bind to the nucleic acid adsorbent thereby separating the nucleic acid from the aqueous liquid fraction or the solution. In anion exchange mixed-mode chromatography, the solid support comprises nucleic acid adsorbent with a positively charged functional group and nucleic acid adsorbent with a further functional group with another type of interaction with the nucleic acid. The functional group and the further functional group may be present within the same nucleic acid adsorbent. For example, a positively charged group functional group can be combined with function group for hydrophobic interactions. An example of a mixed-mode nucleic acid adsorbent is hydroxylapatite (Ca₅(PO₄)₃OH)₂ The solid support may be packed in a column. Alternatively, the solid support may be a free-floating support wherein the particles of the solid support move freely in a container, e.g., batch-binding chromatography. Thus, in one embodiment of the method of the invention, chromatography comprises column chromatography. In another embodiment, chromatography comprises expanded-bed adsorption chromatography. In a preferred embodiment, chromatography comprises batch binding chromatography.

In one embodiment, separating nucleic acids in the method of the present invention comprises batch-binding chromatography comprising: a) adding a nucleic acid adsorbent immobilized to a solid support, preferably to a free-floating solid support, b) preferably stirring or shaking, more preferably shaking with an overhead shaker or agitator c) separating the nucleic acid bound to the nucleic acid adsorbent immobilized to a solid support. In one embodiment, the nucleic acid adsorbent is added to the lysate or the cleared lysate of the method of the invention. In one embodiment, the nucleic acid adsorbent is added to the aqueous liquid fraction, preferably the lipid reduced aqueous liquid fraction, of the method of the invention. In one embodiment, the nucleic acid adsorbent is added to the solution obtained by filtrating the (lipid reduced) aqueous liquid fraction of the method of the invention. In one embodiment, separating the nucleic acid bound to the nucleic acid adsorbent immobilized to a solid support comprises filtration of the nucleic acid bound to the nucleic acid adsorbent immobilized to a solid support. In another embodiment, separating the nucleic acid bound to the nucleic acid adsorbent immobilized to a solid support comprises centrifugation, preferably between about 1 000 g to 4 000 g of the nucleic acid bound to the nucleic acid adsorbent immobilized to a solid support. In a preferred embodiment, separating the nucleic acid bound to the nucleic acid adsorbent immobilized to a solid support comprises sedimentation of the nucleic acid bound to the nucleic acid adsorbent immobilized to a solid support and d) optionally filtration fraction, preferably by dead-end filtration. The method according to the present invention does not contain a step of blending previously separated fractions.

In batch-binding mode chromatography, solid support particles (stationary phase) are added directly to the sample and not, as in column chromatography or expanded bed adsorption chromatography, into a separation column. The binding of the nucleic acids with affinity for the stationary phase to the stationary phase takes place by active mass transfer, diffusion and adsorption in a thoroughly mixed container. The solid support particles are free-floating, e.g., they are homogeneously distributed in the sample (mobile phase) by mixing, stirring, or shaking, e.g., by an overhead shaker, and can be separated from the sample by sedimentation and/or filtration after completion of the mixing process. In batch-binding mode chromatography, in contrast to column chromatography, particles with larger diameters are advantageous due to their faster sedimentation properties. Subsequently, the nucleic acids bound to the solid support can be eluted from the solid support. The elution can be carried out in stages so that different bound nucleic acids can be recovered separately. After elution of the bound nucleic acids and subsequent equilibration, the chromatography material, e.g., the solid support, may be recycled for further use. Batch-binding chromatography enables selective binding of nucleic acids from unpurified, viscous biological samples thereby avoiding cost- and effort-intensive preparation measures of the lysate. Thus, batch-binding chromatography can be directly applied to lysates and may overcome limitations of column chromatography such as the blocking of packed columns by viscous biological samples and the associated disturbances due to a reduced flow rate during the process.

Usually, the nucleic acid concentration in the lysate of step b) of the method of the invention is between about 10% to 15% based on dry matter of the lysate. Separation of nucleic acids as described herein results in a reduction of about 40% or more, preferably about 50% or more, more preferably about 65% or more, more preferably about 75% or more, more preferably about 80% or more relative to the nucleic acid concentration in the lysate while retaining the function properties of the protein.

In a further aspect, the invention provides a protein preparation obtainable by the method according to the method of the invention. In certain embodiments, the protein preparation is characterized as comprising gel forming capacity with about 1% to 10% of the protein preparation per total weight of a solution consisting of the protein preparation and water after heat treatment, preferably without syneresis.

In a preferred embodiment, the protein preparation of the invention is in dry form, preferably in the form of a powder.

In yet another aspect, the invention provides a protein preparation derived from a microorganism, preferably a single cell microorganism, comprising at least about 70% (w/w), or at least about 72%, preferably at least about 75% (w/w), more preferably at least about 85% (w/w) and most preferably at least about 95% (w/w) of protein by dry weight of the protein preparation. In one embodiment, the protein preparation comprises about 70% to 80% (w/w) of protein by dry weight of the protein preparation. In preferred embodiments, the protein preparation comprises native protein.

In a preferred embodiment, the protein preparation of the invention comprises about 110 mg/g or less, preferably about 50 mg/g or less, more preferably about 40 mg/g or less, even more preferably about 20 mg/g or less, even more preferably about 15 mg/g or less of lipid (fat) by dry weight of the protein preparation (assessed using the method for determination of total fat content in cereal products after acid digestion by extraction and gravimetry according to § 64 LFGB L 16.00-5: 2017-10) and optionally about 10% (w/w) or less, preferably about 5.5% (w/w) or less, more preferably about 2.5% nucleic acid by dry weight of the protein preparation. In certain embodiments lipid (fat) by dry weight content (assessed using the method according to § 64 LFGB L 16.00-5: 2017-10) was reduced by about 20% or more (e.g., 25% or more, 30% or more, 35% or more, 40% or more, or 50% or more) following lipid reduction compared to lysate or the aqueous liquid fraction prior to lipid reduction. Moreover, fat-soluble components as measured by the UV method described herein was reduced by about 50-60% or more following lipid reduction compared to lysate or the aqueous liquid fraction prior to lipid reduction. The total lipid (fat) content may be determined, e.g, using the method for determination of total fat content in cereal products after acid digestion by extraction and gravimetry (according to § 64 LFGB L 16.00-5: 2017-10). The designation § 64 LFGB L 16.00-5: 2017-10 describes a method carried out in accordance with DIN standards by a DAkks-certified laboratory (holding an accreditation certificate from the Deutsche Akkreditierungsstelle). The method can be found for example at Beuth Verlag GmbH in the BVL method collection for foods.

In a preferred embodiment, the protein preparation of the invention comprises a water binding capacity of about 4 g/g or more, about 4.5 g/g or more, about 5 g/g or more, about 6.5 or more, such as about 4.5 to 20 g/g, about 4.5 g/g to 10 g/g, about 6 to 10 g/g, about 6 to 7.5 g/g, about 6.0 to 7.0 g/g by dry weight of the protein preparation after heat treatment, preferably at about 80° C. The water binding capacity can be determined by any method known in the art and includes but is not limited to methods as used herein in the Examples, or as described in Kneifel, W. and Seiler, A. (1993) “Water-holding Properties of Milk Protein Products—A Review”, Food Structure: Vol. 12: No. 3, Article 3; or as described in Wang, J. S., Wang, A. B., Zang, X. P., Tan, L., Xu, B. Y., Chen, H. H., et al. (2019). “Physicochemical, functional and emulsion properties of edible protein from avocado (Persea americana Mill.) oil processing by-products.”, Food Chemistry, 288 (February), 146-153; or as described in S. Thammakiti, M. Suphantharika, T. Phaesuwan, C. Verduyn: “Preparation of spent brewer's yeast β-glucans for potential applications in the food industry, Int. J. Food Sci. Technol. 39 (2004) 21-29. This method adapted in Vlatka Petravic-Tominac, Vesna Zechner-Krpan, Katarina Berkovic, Petra Galovic, Zoran Herceg, Sinisa Srecec, Igor Spoljaric: “Rheological Properties, Water-Holding and Oil-Binding Capacities of Particulate β-Glucans Isolated from Spent Brewer's Yeast by Three Different Procedures”, January 2011, Food Technology and Biotechnology 49(1):56-64), all incorporated herein by reference. In particularly preferred embodiment, the water binding capacity is determined by a method comprising:
i) preparing a solution comprising 0.5 g protein preparation to be tested in 4 mL or 5 mL demineralized water (w/v),
ii) mixing the solution for 20 sec.,
iii) repeating mixing seven times in time intervals of 10 min.,
iv) placing the solution at 80° C. for 10 min.,
v) cooling the solution down to room temperature,
vi) centrifuging at 2000 g for 25 min. at 20° C. in a container,
vii) discarding the supernatant and obtaining a sample saturated with water,
viii) removing residual water from the container by positing the container in an angle of 20° for 10 min., and
ix) calculation of the water binding capacity (WBC) by (weight of the sample saturated with water−weight of the container−protein mass)/protein mass.

In a preferred embodiment, the protein preparation of the invention comprises a gel forming capacity, preferably without syneresis, with about 0.5%, 1%, 1.5%, 2%, 2.5%, 3%, 3.5%, 4%, 4.5%, 5%, 5.5% or 10% or more, preferably about 1%, 1.5%, 2%, 2.5%, 3%, 3.5%, 4%, 4.5% or 5% or more of the protein preparation per total weight of a solution consisting of protein preparation and water after heat treatment, preferably at about 80° C. Preferably the gel forming is observed with about 1% to 20%, 5% to 20%, preferably about 1 to 10%, about 2% to 10%, about 5 to 10% or about 1 to 7% or about 7% to 10% of the protein preparation per total weight of a solution consisting of the protein preparation and water after heat treatment. In certain embodiments the protein preparation of the invention comprises a gel forming capacity, preferably without syneresis, already with about 2%, 2.5%, 3%, 3.5%, 4%, 4.5%, 5%, 5.5% and/or about 10% (w/w) of the protein preparation per total weight of a solution consisting of protein preparation and water after heat treatment, preferably with (as little as) about 2%, 2.5%, 3%, 3.5%, 4%, 4.5%, 5%, or 5.5% (w/w). The gel forming capacity can be determined by any method known in the art and includes but is not limited to the methods as used herein in the Examples or as described in Langton et al., “Gelation of faba bean proteins—Effect of extraction method, pH and NaCl”, Food Hydrocolloids (2020) 103, 105622:1-8) which is incorporated herein by reference. In a particularly preferred embodiment, the gel forming capacity is determined by a method comprising:
i) preparing a solution comprising 5% protein preparation to be tested in demineralized water (w/w) and stirring the solution of 20 min.,
ii) subjecting the solution to 80° C. for 20 min.,
iii) determining the state of the preparation to be tested wherein gel formation is present if the state of the preparation to be tested is comparable to the state of a reference preparation comprising 5% egg white protein.

In one embodiment, the protein preparation of the invention comprises an oil binding capacity of about 0.3 g/g or more, preferably about 0.5 g/g or more, about 2 g/g or more, more preferably 3 g/g or more or about 0.3 g/g to 4 g/g, about 0.5 g/g to 4 g/g, about 0.5 g/g to 3 g/g, about 0.5 g/g to 2 g/g or about 0.5 to 0.7 g/g by dry weight of the protein preparation after heat treatment, preferably at about 80° C. The oil binding capacity can be determined by any method known in the art and includes but is not limited to methods as used herein in the Examples, or as described in Wang, J. S., Wang, A. B., Zang, X. P., Tan, L., Xu, B. Y., Chen, H. H., et al. (2019). “Physicochemical, functional and emulsion properties of edible protein from avocado (Persea americana Mill.) oil processing by-products.”, Food Chemistry, 288 (February), 146-153; or as described in Vlatka Petravic et al. “Rheological Properties, Water-Holding and Oil-Binding Capacities of Particulate β-Glucans Isolated from Spent Brewer's Yeast by Three Different Procedures”, January 2011, Food Technology and Biotechnology 49(1):56-64; or as described in V. PETRAVI-TOMINAC et al.: “Properties of b-Glucans from Brewer's Yeast”, Food Technol. Biotechnol. 49 (1) 56-64 (2011), or as described in Zayas, J. F.: Oil and Fat Binding Properties of Proteins. In: Functionality of Proteins in Food, Springer Verlag Berlin Heidelberg, 1997, all incorporated herein by reference. In a particularly preferred embodiment, the oil binding capacity is determined by a method comprising:
i) preparing an dispersion to be tested comprising 0.5 g protein preparation in 4 mL or 5 mL sunflower oil (w/v),
ii) mixing the dispersion for 20 sec.,
iii) repeating mixing seven times in time intervals of 10 min.,
iv) placing the dispersion at 80° C. for 10 min.,
v) cooling the dispersion down to room temperature,
vi) centrifuging the dispersion at 2000 g for 25 min. at 20° C. in a container,
vii) discarding the supernatant and obtaining a sample saturated with oil,
viii) removing of residual oil from the container by positing the container in an angle of 20° for 10 min.,
ix) calculation of the oil binding capacity (OBC) by (weight of the sample saturated with oil−weight of the container−protein mass)/protein mass.

In one embodiment, the protein preparation of the invention comprises a powder solubility of about 74% or more, about 75% or more, about 78% or more, about 80% or more, about 82% or more, about 85% or more of the initial concentration of the protein preparation suspended in water by total weight of the suspension. The powder solubility can be determined by any method known in the art and includes but is not limited to methods as used herein in the Examples, or as described in U.S. Pat. No. 4,465,702, or as described in Elif Ezgi Ozdemir, Ahmet Gorgog, Esra Gençdag, Fatih Mehmet Yilmaz., “Physicochemical, functional and emulsifying properties of plant protein powder from industrial sesame processing waste as affected by spray and freeze drying.”, LWT Food Science and Technology, 154 (2022) 112646, all incorporated by reference herein. In a preferred embodiment, the powder solubility is determined as disclosed in U.S. Pat. No. 4,465,702 which is incorporated herein by reference. In particularly preferred embodiment, the powder solubility is determined by a method comprising:
i) preparing a solution to be tested comprising 2% protein preparation in 50 ml demineralized water (w/v),
ii) mixing the solution, preferably at 800 rpm, with a magnetic stirrer for 30 min.,
iii) centrifuging at 2 000 g for 25 min. at 20° C.,
iv) transferring 25 ml of the supernatant into an aluminum shell,
v) drying the supernatant for 1.5 h at 160° C. to obtain a sample,
vi) cooling down of the aluminum shell with the sample in a desiccator
vii) weighing of the aluminum shell with the sample,
viii) calculation of the powder solubility (%) by (weight of the sample with aluminum shell−empty weight of the aluminum shell)/(protein weight of the solution×concentration of the protein preparation)×2×100%.

In one embodiment, the protein preparation of the invention comprises an emulsion activity of about 54% or more, about 55% or more, about 56% or more, about 57% or more, about 58% or more, about 59% or more, about 60% or more per total volume of an emulsion consisting of 25 ml sunflower oil and 25 ml solution consisting of 5% protein preparation in water after centrifugation, wherein the emulsion activity is defined as emulsion layer (ml)/total volume (ml)×100%. The emulsion activity can be determined by any method known in the art and includes but is not limited to methods as used herein in the Examples, turbidimetry as described in Pearce et al., “Emulsifying properties of proteins: evaluation of a turbidimetric technique”, J. Agric. Food, Chem., 198, 26:716-723, or as described in Elif Ezgi Ozdemir, Ahmet Gorgog, Esra Gençdag, Fatih Mehmet Yilmaz, “Physicochemical, functional and emulsifying properties of plant protein powder from industrial sesame processing waste as affected by spray and freeze drying”, LWT Food Science and Technology, 154 (2022) 112646, or as described in Lam and Nickerson, Food proteins: A review on their emulsifying properties using a structure-function approach. Food Chemistry 141 2013: 975-984, or as described in Hasenhuettl and Hartel, Food emulsifiers and their applications: Second edition, 2008, Springer Science+Business Media, LLC, all incorporated by reference herein. In a preferred embodiment, the emulsion activity is determined by the method disclosed Ozdemir et al which is incorporated herein by reference. In particularly preferred embodiment, the emulsion activity is determined by a method comprising:
i) preparing an emulsion to be tested consisting of 25 ml of 5% protein preparation powder solution in water (w/v) and 25 ml sunflower oil,
ii) homogenizing the emulsion,
iii) immediately centrifuging at 1 200 g for 5 min.,
iv) measuring the emulsion layer and the total volume
v) calculating the emulsion activity by emulsion layer (ml)/total volume (ml)×100%.

In one embodiment, the protein preparation of the invention comprises an emulsion stability of about 97% or more, preferably about 98% or more, more preferably about 100% per total volume of an emulsion consisting of 25 ml sunflower oil and 25 ml solution consisting of 5% protein preparation in water after subjecting the emulsion to 80° C. for 30 min. and subsequent centrifugation, wherein the emulsion stability is defined as emulsion layer (ml)/total volume (ml)×100%. The emulsion stability can be determined by any method known in the art and includes but is not limited to methods as used herein in the Examples, turbidimetry as described in Pearce et al., “Emulsifying properties of proteins: evaluation of a turbidimetric technique”, J. Agric. Food, Chem., 198, 26:716-723, or as described in Elif Ezgi Ozdemir, Ahmet Gorgog, Esra Gençdag, Fatih Mehmet Yilmaz, “Physicochemical, functional and emulsifying properties of plant protein powder from industrial sesame processing waste as affected by spray and freeze drying”, LWT Food Science and Technology, 154 (2022) 112646, all incorporated by reference herein. In a preferred embodiment, the emulsion stability is determined as disclosed Ozdemir et al which is incorporated herein by reference. In particularly preferred embodiment, the emulsion activity is determined by a method comprising:
i) preparing an emulsion to be tested consisting of 25 ml of a 5% protein preparation powder solution in water (w/v) and 25 ml sunflower oil,
ii) homogenizing the emulsion,
iii) centrifuging at 1 200 g for 5 min.,
iv) subjecting the emulsion to 80° C. for 30 min.
v) cooling the emulsion,
vi) centrifuging at 1 200 g for 5 min.,
vii) measuring the emulsion layer and the total volume
viii) calculating the emulsion stability by emulsion layer (ml)/total volume (ml)×100%.

In one embodiment, the protein preparation of the invention comprises a foaming capacity of about 40% or more, about 41% or more, about 42% or more, about 43% or more, about 44% or more, about 45% or more, about 46% or more, about 47% or more, about 48% or more, about 49% or more, about 50% or more, about 51% or more, about 52% or more of foam volume per total volume of a solution consisting of 100 mg protein preparation in 10 ml water, wherein foam capacity is defined as foam volume (ml)/total volume (ml)×100%. The foaming capacity can be determined by any method known in the art and includes but is not limited to methods as used herein in the Examples, turbidimetry as described in Pearce et al., “Emulsifying properties of proteins: evaluation of a turbidimetric technique”, J. Agric. Food, Chem., 198, 26:716-723, or as described in Elif Ezgi Ozdemir, Ahmet Gorgüç, Esra Gençdag, Fatih Mehmet Yilmaz, “Physicochemical, functional and emulsifying properties of plant protein powder from industrial sesame processing waste as affected by spray and freeze drying”, LWT Food Science and Technology, 154 (2022) 112646, or as described in Richard K Owusu-Apenten, “Testing protein functionality”, April 2004, in “Proteins in food processing”: 217-244, 1. edition, Publisher: Woodhead Publishing Ltd, Editors: Yada, or as described in “Proteinschaume in der Lebensmittelproduktion: Mechanismenaufklärung, Modellierung und Simulation”, 2014: 1-149, Teilprojekt 3: Charakterisierung der Struktur und Dynamik von proteinstabilisierten Schaumen (AiF 17124 N), editor: Forschungskreis der Ernährungsindustrie e.V., Bonner Universitäts-Buchdruckerei, Bonn, or foam durability method as described in MEBAK online. Methode B-420.09.100. Schaumhaltbarkeit nach ROSS und CLARK. Rev. 2020-10. Mitteleuropäische Brautechnische Analysenkommission (MEBAK®) e.V., Freising. https://www.mebak.org/methode/b-420-09-100/schaumhaltbarkeit-nach-ross-und-clark/711 all incorporated by reference herein. In a preferred embodiment, the foaming capacity is determined by a method comprising:
i) providing a dispersion of 100 mg of the protein preparation to be tested in 10 ml distilled water,
ii) homogenizing the dispersion for 30 sec., preferably at 11 000 rpm,
iii) transferring the dispersion into a measuring cylinder,
iv) measuring foam volume after 30 sec.,
iv) calculating the foam capacity by foam volume (ml)/total volume (ml)×100%.

In one embodiment, the protein preparation of the invention comprises a foaming stability of about 20% or more, about 30% or more, about 40% or more, about 50% or more, about 60% or more, about 70% or more, about 75% or more, about 76% or more of foam volume per total volume of a solution consisting of 100 mg protein preparation in 10 ml water after 60 minutes, wherein foam stability is defined as foam volume (ml) after 60 minutes/initial volume (ml)×100%. The foaming stability can be determined by any method known in the art and includes but is not limited to methods as used herein in the Examples, or turbidimetry as described in Pearce et al., “Emulsifying properties of proteins: evaluation of a turbidimetrictechnique”, J. Agric. Food, Chem., 198, 26:716-723, or the method as described in Elif Ezgi Ozdemir, Ahmet Gorgog, Esra Gençdag, Fatih Mehmet Yilmaz, “Physicochemical, functional and emulsifying properties of plant protein powder from industrial sesame processing waste as affected by spray and freeze drying”, LWT Food Science and Technology, 154 (2022) 112646, or as described in Richard K Owusu-Apenten, “Testing protein functionality”, April 2004, in “Proteins in food processing”: 217-244, 1. edition, Publisher: Woodhead Publishing Ltd, Editors: Yada, or the method as described in “Proteinschaume in der Lebensmittelproduktion: Mechanismenaufklärung, Modellierung und Simulation”, 2014: 1-149, Teilprojekt 3: Charakterisierung der Struktur und Dynamik von proteinstabilisierten Schaumen (AiF 17124 N), editor: Forschungskreis der Ernährungsindustrie e.V., Bonner Universitäts-Buchdruckerei, Bonn, or foam durability method as described in MEBAK online. Methode B-420.09.100. Schaumhaltbarkeit nach ROSS und CLARK. Rev. 2020-10. Mitteleuropäische Brautechnische Analysenkommission (MEBAK®) e.V., Freising. https://www.mebak.org/methode/b-420-09-100/schaumhaltbarkeit-nach-ross-und-clark/711 all incorporated by reference herein. In a preferred embodiment, the foaming stability is determined by a method comprising:
i) providing a dispersion of 100 mg of the protein preparation to be tested in 10 ml distilled water,
ii) homogenizing the dispersion for 30 sec. preferably at 11 000 rpm,
iii) transferring the dispersion into a measuring cylinder,
iv) measuring foam volume after 60 min.,
v) calculating the foam stability by foam volume (ml) measured after 60 minutes/initial volume (ml)×100%. The initial volume is the volume measured directly after transferring the dispersion into the measuring cylinder.

In a preferred embodiment of the protein preparation of the invention, the protein preparation of the invention is in dry form, preferably in the form of a powder.

In yet a further preferred embodiment of the protein preparations of the invention, the microorganism is a fungus, preferably a fungus as described herein, more preferably Aspergillus niger.

In yet a further preferred embodiment of the protein preparation of the invention, the microorganism is an alga, preferably an alga as described herein, more preferably an alga selected from the group consisting of Arthrospira maxima (Spirulina maxima), Arthospira platensis (Spirulina platensis), Chlorella vulgaris and Euglena gracilis.

In a particular preferred embodiment of the protein preparation of the invention, microorganism is a yeast, preferably a yeast as described herein, more preferably a yeast selected from the group consisting of Saccharomyces spp., preferably S. cerevisiae, S. carlsbergensis, S. bayanus, S. ellipsoides, S. uvarum, S. ludwigii or S. pastorianus, Pichia spp., preferably P. pastoris; Hansenula spp.; Candida spp., preferably C. utilis; Torulopsis spp.; and Yarrowia lipolytica.

In another aspect, the invention provides a method for preparing a protein gel comprising:
(a) providing a protein preparation according to the invention,
(b) mixing the protein preparation with an aqueous carrier fluid, and
(c) heating the mixture to a temperature of at least about 55° C. to provide the protein gel.

In one embodiment, the protein preparation is provided in a solution. In a preferred embodiment, the protein preparation is provided in dried form, e.g., as a powder. In one embodiment, the aqueous carrier fluid includes but is not limited to water or an aqueous carrier fluid, e.g., a buffer. In one embodiment, the protein preparation is present at about 5% (w/w) or more, about 6% or more, about 7% or more, about 8% or more, about 9% or more, about 10% or more, about 20% or more, about 30% or more, about 40% or more, about 50% or more, preferably about 5% to about 20%, more preferably about 5% to 10% per weight relative to the total volume of the aqueous carrier fluid. Mixing the protein preparation with an aqueous carrier fluid can be carried out by stirring or shaking. In one embodiment, the mixture is heated to a temperature of at least about 55° C., at least about 60° C., at least about 65° C., at least about 70° C., at least about 75° C., at least about 80° C. At this temperature, the protein denatures and forms a gel. Heating can be performed in a water bath.

In yet another aspect, the invention relates to the use of the protein preparation of the invention for preparing a food product, preferably for human or animal use, or a dietary supplement. In one embodiment, the protein preparation acts as equivalent or substitute of methylcellulose, particularly in meat substitutes, ice cream, bakery products, cake cream, mayonnaise, instant food products or frozen products. In another embodiment, the protein preparation acts as equivalent or substitute of plant protein, egg protein, preferably an egg yolk protein and/or an egg white protein, meat protein, gluten protein and/or a milk protein. In a preferred embodiment, the protein preparation acts as equivalent or substitute of egg protein, preferably an egg yolk protein and/or an egg white protein, e.g., in bakery products, pasta, savory systems e.g., scrambled eggs, omelet, mayonnaise, or dressings. In a preferred embodiment, the protein preparation acts as equivalent or substitute of meat protein, e.g., in nuggets, steak, minced-meat, burger patties, kebab, or gyros. In a preferred embodiment, the protein preparation acts as equivalent or substitute of milk protein, e.g., in milk, fermented drinks, dairy products, spoonables, e.g., yoghurt, mousses, cream, cake cream, quark, or ice cream. In a preferred embodiment, the protein preparation acts as equivalent or substitute of gluten protein, e.g., in bakery products or dough products, e.g., pasta. The protein preparation of the invention may also be used in protein-enriched systems, e.g., muesli, protein bars, bread, bakery products or dough products. The protein preparation of the invention may also be used in combination with other proteins, e.g., non-animal proteins, such as plant proteins, e.g., rice protein, pea protein, sunflower protein, soy protein, hemp protein, faba bean protein egg protein or potato protein, or animal proteins such as meat protein, fish protein, insect protein, egg protein or milk protein. In a further embodiment, the protein preparation acts as gelling agent, foaming agent, texturing agent, binding agent, thickening agent, stabilizing agent and/or emulsifying agent. The functional properties, e.g., water binding capacity, gel forming capacity, powder solubility, oil binding capacity, emulsion activity or stability, foaming activity or stability are described elsewhere herein and in the Examples. In a preferred embodiment, the protein preparation acts as gelling agent, preferably with a water binding capacity of about 4 g/g or more, preferably about 5 g/g or more, more preferably about 6 to 7 g/g by dry weight of the protein preparation after heat treatment. In a further preferred embodiment, protein preparation of the invention is used for preparing a food product or a dietary supplement which is a vegan or a non-animal derived food product or dietary supplement. In a further preferred embodiment, protein preparation of the invention is used for preparing a food product or a dietary supplement which is a food product and/or a dietary supplement without rancid tase.

In a further aspect, the invention provides a dietary supplement comprising the protein preparation of the invention. In one embodiment, the dietary supplement is in the form of a tablet, pill, powder, granulate, or flake.

In a further aspect, the invention provides a food product comprising the protein preparation of the invention or the dietary supplement of the invention. In one embodiment, the food product is a meat substitute, an egg substitute, a fish substitute, an insect substitute or a dairy product substitute, preferably a non-animal derived substitute. In another embodiment, the food product is a nugget, burger patties, kebab, steak, minced meat, gyros, milk, fermented drink, a dairy product, a spoonable, e.g., yoghurt, mousse, cream, cake cream, quark, or ice cream; a bakery product, a dough product, pasta, a savory system e.g., scrambled egg, omelet, mayonnaise, or dressing; muesli, or a protein bar.

In a further aspect, the invention provides a method of preparing a food product or dietary supplement product without a rancid taste comprising:
a) providing a protein preparation according to the invention,
b) optionally mixing the protein preparation with one or more of further ingredients of said product or supplement, and
c) preparing the food product of the invention or the dietary supplement or the invention.

In this embodiment, the rancid taste of the food product or dietary supplement is reduced or absent because the protein preparation of the invention lacks lipids, preferably unsaturated lipids, e.g., oleic acid, palmitoleic acid, and linoleic acid. These unsaturated lipids are particularly susceptible to oxidation processes and therefore become rancid more quickly than saturated fatty acids.

In one embodiment the further ingredient may comprise ingredients of bakery products, dairy products, dough products, egg products or meat products.

In a further aspect, the invention provides a method of preparing a food product comprising the use of the protein preparation of the invention wherein the protein preparation is a) a gelling agent, preferably with a gel forming capacity as described herein, b) a methylcellulose substitute, c) a plant protein substitute, d) meat protein substitute, e) gluten protein substitute, f) milk protein substitute, g) fish protein substitute or f) an egg protein substitute, preferably an egg yolk protein substitute and/or an egg white protein substitute.

In a further aspect, the invention provides a method of preparing a dietary supplement comprising the use of the protein preparation of the invention wherein the protein preparation is a) a gelling agent, preferably with a gel forming capacity as described herein, or b) a methylcellulose substitute.

In a further aspect, the invention provides a method of obtaining native protein of a microorganism comprising:
a) providing a microorganism, and optionally subjecting the microorganism to one or more pre-treatment step(s),
b) lysing the microorganism thereby preparing a lysate comprising an aqueous liquid fraction comprising nucleic acid and solved native protein of the microorganism,
c) separating the nucleic acid from the aqueous liquid fraction comprising:
   i) adding a nucleic acid adsorbent immobilized to a solid support, preferably to a free-floating solid support to the aqueous liquid fraction,
   ii) optionally stirring or shaking, preferably by an overhead shaker or agitator, and
   iii) separating the nucleic acid bound to the nucleic acid adsorbent immobilized to a solid support, preferably by sedimentation and optionally filtration, preferably by dead-end filtration,
   thereby obtaining an aqueous liquid fraction wherein the aqueous liquid fraction is a nucleic acid reduced aqueous liquid fraction,
d) filtrating the (nucleic acid reduced) aqueous liquid fraction thereby obtaining a solution comprising purified solved native protein of the microorganism and an aqueous solvent, preferably water or a saline solution, and
e) optionally removing at least a part of the aqueous solvent,
f) optionally sterilizing the solution thereby obtaining a sterilized solution comprising purified solved native protein of the microorganism and an aqueous solvent, and
g) optionally removing at least a part of the aqueous solvent of the sterilized solution of step f).

As far as applicable the specific embodiments as disclosed herein in the context of the methods of the invention, particularly the specific embodiments for steps a), b), c) of separating lipid, d), e), f) and g) as disclosed herein also apply to this aspect of the invention.

The type of microorganism that is used in the present invention is not specially limited. In one embodiment, the microorganism is a eukaryotic microorganism. In a further embodiment, the microorganism is a eukaryotic microorganism selected from the group consisting of a fungus; a yeast, and an alga. In a preferred embodiment, the microorganism is a fungus, preferably Aspergillus niger. In a further preferred embodiment, the microorganism is an alga, preferably an alga selected from the group consisting of selected from the group consisting of Arthrospira maxima (Spirulina maxima), Arthospira platensis (Spirulina platensis), Chlorella vulgaris and Euglena gracilis. In a particularly preferred embodiment, the yeast is selected from the group consisting of Saccharomyces spp., preferably S. cerevisiae, S. carlsbergensis, S. bayanus, S. ellipsoides, S. uvarum, S. ludwigii or S. pastorianus, Pichia spp., preferably P. pastoris; Hansenula spp.; Candida spp., preferably C. utilis; Torulopsis spp.; and Yarrowia lipolytica. In a most preferred embodiment, the microorganism is a yeast of Saccharomyces spp., more preferably from S. cerevisiae, S. pastorianus or S. carlsbergensis. In another embodiment the microorganism is a prokaryotic microorganism. In a preferred embodiment, the microorganism is a bacterium, preferably a bacterium selected from the group consisting of selected from the group consisting of Bacillus subtilis, Lactobacillus spp., Corynebacterium glutamicum, Methylomonas spp., Spirulina ssp., and Xanthomonas spp. In one embodiment, the microorganism is a single cell organism.

In a further embodiment, the method of the invention comprises a step b1) of clearing the lysate, preferably by centrifugation. Alternatively, the method of the invention comprises a step c1) of clearing the aqueous liquid fraction, preferably the nucleic acid reduced aqueous liquid fraction, preferably by centrifugation. In preferred embodiment, separating nucleic acid in step c) is performed after step b), i.e., from the lysate, or b1), i.e., after clearing the lysate. In a further embodiment, step c) of separating nucleic acids can be performed after step d), i.e., after filtrating the aqueous liquid fraction from the solution. In one embodiment, the nucleic acid adsorbent is added to the lysate. In one embodiment, stirring or shaking is performed by an overhead shaker. In one embodiment, the nucleic acid adsorbent is added to the solution of the method of the invention. In one embodiment, separating the nucleic acid bound to the nucleic acid adsorbent immobilized to a solid support comprises filtration of the nucleic acid bound to the nucleic acid adsorbent immobilized to a solid support. In another embodiment, separating the nucleic acid bound to the nucleic acid adsorbent immobilized to a solid support comprises centrifugation, preferably between about 1 000 g to 4 000 g of the nucleic acid bound to the nucleic acid adsorbent immobilized to a solid support. In a preferred embodiment, separating the nucleic acid bound to the nucleic acid adsorbent immobilized to a solid support comprises sedimentation of the nucleic acid bound to the nucleic acid adsorbent immobilized to a solid support and d) optionally filtration, preferably by dead-end filtration.

For the purpose of the present invention, separating nucleic acid is performed at a temperature of about 40° C. or less, about 30° C. or less, about 20° C. or less, or about 10° C. or less (up to a temperature of about 2° C. to 8° C.), preferably about 30° C. or less and/or does not involve the use of low or high pH conditions to avoid denaturation of the protein and loss of the functional properties of the protein. In a preferred embodiment, steps b) to g), preferably steps b) to d) of the method, are performed at a temperature of about 40° C. or less, preferably about 30° C. or less, such as about 30° C. to 2° C., preferably about 30° C. to 8° C., more preferably about 30° C. to 20° C.

The separation of nucleic acids can be carried out in any volume, from lab scale to industrial scale. For example, the chromatography may be carried out in a volume of about 0.5 l to 2 l. The chromatography may also be carried out in a volume of about 1 l or more, 10 l or more, 20 l or more 50 l or more, 100 l or more, 200 l or more, 500 l or more, 1 000 l or more, 2 000 l or more, 5 000 l or more, 7 000 l or more, or 10 000 l or more.

In a preferred embodiment, separating the nucleic acid from the aqueous liquid fraction is performed by chromatography, preferably anion-exchange chromatography, or anion exchange mixed mode chromatography. In anion-exchange chromatography, a nucleic acid adsorbent is immobilized on a solid support, for example ceramic or resin, e.g., styrene-DVB. The nucleic acid adsorbent comprises a positively charged functional group, e.g., a quaternary ammonium compound. Nucleic acids with a negatively charged backbone can bind to the nucleic acid adsorbent thereby separating the nucleic acid from the aqueous liquid fraction or the solution. In anion exchange mixed-mode chromatography, the solid support comprises nucleic acid adsorbent with a positively charged functional group and nucleic acid adsorbent with a further functional group with another type of interaction with the nucleic acid. The functional group and the further functional group may be present within the same nucleic acid adsorbent. For example, a positively charged group functional group can be combined with function group for hydrophobic interactions. An example of a mixed-mode nucleic acid adsorbent is hydroxylapatite (Ca₅(PO₄)₃OH)₂ In a preferred embodiment, the solid support is a free-floating support wherein the particles of the solid support move freely in a container, e.g., batch-binding chromatography.

In a further preferred embodiment, the method of the invention further comprises a step of separating lipid from the aqueous liquid fraction as described herein in the context of the methods of the invention. The specific embodiments of separating lipids as disclosed herein are fully applicable to the method of separating nucleic acids. In this embodiment, the further step of separating lipids can be performed prior to step c) of separating nucleic acids, i.e., from the lysate or the cleared lysate of step b1); after step c) i.e., from the nucleic reduced aqueous liquid fraction comprising the solved native protein of the microorganism, or after step c1) of clearing the aqueous liquid fraction.

The invention is further characterized by the following items:
1. A method of preparing native protein of a microorganism comprising:
   a) providing the microorganism, and optionally subjecting the microorganism to one or more pre-treatment step(s),
   b) lysing the microorganism thereby obtaining a lysate comprising an aqueous liquid fraction comprising lipid and solved native protein of the microorganism, further comprising a step b1) of clearing the lysate, preferably by centrifugation or filtration,
   c) separating the lipid from the aqueous liquid fraction using mechanical means thereby obtaining an aqueous liquid fraction wherein the aqueous liquid fraction is a lipid reduced aqueous liquid fraction comprising the solved native protein of the microorganism,
   d) filtrating the aqueous liquid fraction thereby obtaining a solution comprising purified solved native protein of the microorganism and an aqueous solvent, preferably water or a saline solution,
   e) optionally removing at least a part of the aqueous solvent,
   f) optionally sterilizing the solution thereby obtaining a sterilized solution comprising purified solved native protein of the microorganism and an aqueous solvent, and
   g) optionally removing at least a part of the aqueous solvent of the sterilized solution.
2. The method of item 1 wherein separating the lipid from the aqueous liquid fraction using mechanical means in step c) is based on different density of the lipid and the lipid reduced aqueous liquid fraction comprising the solved native protein of the microorganism (i.e., wherein separating the lipid is performed using a centrifugal three-phase separator).
3. The method of item 1 or 2 wherein separating the lipid from the aqueous liquid fraction by mechanical means in step c) is performed by a centrifugal separator (i.e., a centrifugal three-phase separator), preferably a skimming separator and/or a three-phase decanter.
4. The method of any one of the preceding items wherein filtrating in step d) is ultrafiltration, preferably diafiltration/ultrafiltration, preferably with a molecular weight cut-off in a range of about 1 kDa to about 100 kDa, preferably of about 3 kDa to about 50 kDa, more preferably of about 5 kDa to about 15 kDa, most preferably of about 10 kDa.
5. The method of any one of the preceding items wherein the microorganism is a eukaryotic microorganism, preferably a eukaryotic microorganism selected from the group consisting of a fungus, preferably Aspergillus niger, a yeast, and an alga, preferably Arthrospira maxima (Spirulina maxima), Arthospira platensis (Spirulina platensis), Chlorella vulgaris or Euglena gracilis.
6. The method of any one of the preceding items wherein the microorganism is a yeast selected from the group consisting of Saccharomyces spp., preferably S. cerevisiae, S. carlsbergensis, S. bayanus, S. ellipsoides, S. uvarum, S. ludwigii or S. pastorianus, Pichia spp., preferably P. pastoris; Hansenula spp.; Candida spp., preferably C. utilis; Torulopsis spp.; and Yarrowia lipolytica.
7. The method of any one of the items 1 to 4 wherein the microorganism is a prokaryotic microorganism, particularly a bacterium selected from the group consisting of Bacillus subtilis, Lactobacillus spp., Corynebacterium glutamicum, Methylomonas spp., Spirulina ssp., and Xanthomonas spp.
8. The method of any one of the preceding items wherein the microorganism is a single-cell organism.
9. The method of any one of the preceding items wherein the lysing in step b) comprises mechanical lysis, preferably mechanical lysis comprising high pressure homogenization and/or bead milling.
10. The method of any one of items 1 to 8 wherein the lysing in step b) comprises physical lysis, preferably physical lysis comprising sonoporation and/or electroporation.
11. The method of any one of the preceding items wherein the method comprises a step b1) of clearing the lysate by centrifugation.
12. The method of any one of items 1 to 10 wherein the method further comprises a step c1) of clearing the aqueous liquid fraction, preferably by centrifugation.
13. The method of any one of the preceding items wherein steps b) to g), preferably steps b) to d), are performed at a temperature of about 40° C. or less, preferably at a temperature in the range of about 30° C. to about 20° C.
14. The method of any one of the preceding items wherein the one or more pre-treatment step(s) in step a) are selected from the group consisting of filtering, sieving, washing, and centrifugation of the microorganism.
15. The method of any one of the preceding items wherein sterilizing in step f) is performed by sterile filtration.
16. The method of any one of the preceding items wherein removing at least a part of the solvent in step e) and/or step g) comprises removing at least about 50% of the solvent, preferably at least about 75%, more preferably at least about 90%, most preferably at least about 94%.
17. The method of any one of the preceding items wherein removing at least a part of the solvent in step e) and/or step g) comprises spray-drying, vacuum-drying, drum-drying, fluidized bed drying or freeze-drying.
18. The method of any one of the preceding items wherein the method comprises a further step of separating nucleic acid from the aqueous liquid fraction, preferably from the lipid reduced aqueous liquid fraction comprising the solved native protein of the microorganism of step c) or the solution.
19. The method of item 18 wherein separating nucleic acid comprises chromatography, wherein the chromatography is anion exchange chromatography and/or anion exchange mixed-mode chromatography.
20. The method of item 19 wherein chromatography comprises using a nucleic acid adsorbent immobilized on a solid support, preferably wherein the nucleic acid adsorbent comprises a quaternary ammonium compound as functional group.
21. The method of item 19 or 20 wherein chromatography comprises a) column chromatography, preferably in an expanded-bed adsorption mode, or b) batch-binding chromatography.
22. The method of item 21 wherein chromatography performed in a batch-binding mode comprises: a) adding a nucleic acid adsorbent immobilized to a solid support, preferably a free-floating solid support, b) preferably stirring or shaking, c) separating the nucleic acid bound to the nucleic acid adsorbent immobilized to a solid support, preferably by sedimentation and optionally filtration.
23. A protein preparation obtainable by the method according to any one of items 1 to 22 wherein preferably the protein preparation comprises a gel forming capacity with about 1% to 10% of the protein preparation per total weight of a solution consisting of the protein preparation and water after heat treatment, preferably without syneresis, and optionally:
   a) at least about 70% (w/w), preferably at least about 75% (w/w), more preferably at least about 85% (w/w) and most preferably at least about 95% (w/w) of protein by dry weight of the protein preparation,
   b) about 110 mg/g or less, preferably about 50 mg/g or less, more preferably about 40 mg/g or less, even more preferably about 20 mg/g or less, even more preferably 15 mg/g or less of lipid by dry weight of the protein preparation,
   c) a water binding capacity of about 4 g/g or more, preferably about 5 g/g or more, more preferably about 6.5 g/g by dry weight of the protein preparation after heat treatment,
   d) an oil binding capacity of about 0.3 g/g or more, preferably about 0.5 g/g or more, more preferably about 0.5 to 0.7 g/g by dry weight of the protein preparation after heat treatment,
   e) a powder solubility of about 74% or more, preferably about 80% or more, of the initial concentration of the protein preparation suspended in water by total weight of the suspension,
   f) a gel forming capacity with about 2%, about 3%, about 4%, about 5% or about 5.5% of the protein preparation per total weight of a solution consisting of protein preparation and water after heat treatment, preferably without syneresis,
   g) an emulsion activity of about 55% or more, preferably about 56% or more and more preferably 57% or more per total volume of an emulsion consisting of 25 ml sunflower oil and 25 ml solution consisting of 5% protein preparation powder solution in water (w/v) after centrifugation, wherein the emulsion activity is defined as emulsion layer (ml)/total volume (ml)×100%,
   h) an emulsion stability of about 97% or more, preferably about 98% or more, more preferably about 100% or more per total volume of an emulsion consisting of 25 ml sunflower oil and 25 ml solution consisting of 5% protein preparation powder solution in water (w/v) after subjecting the emulsion to 80° C. for 30 min. and subsequent centrifugation, wherein the emulsion stability is defined as emulsion layer (ml)/total volume (ml)×100%,
   i) a foaming capacity of about 40% or more, preferably about 48% or more and more preferably about 50% or more of foam volume per total volume of a dispersion consisting of 10 ml water in 100 mg protein preparation, wherein foam capacity is defined as foam volume (ml)/total volume (ml)×100%,
   j) a foaming stability of about 20% or more, preferably about 40% or more and more preferably about 75% or more of foam volume per total volume of a dispersion consisting of 10 ml water in 100 mg protein preparation after 60 minutes, wherein foam stability is defined as foam volume (ml) measured after 60 minutes/initial volume (ml)×100%, and/or
   k) about 10% (w/w) or less, more preferably about 5.5% (w/w) or less, even more preferably 2.5% (w/w) or less nucleic acid by dry weight of the protein preparation.
24. The protein preparation of item 23 wherein the protein preparation is in dry form, preferably in the form of a powder.
25. A protein preparation derived from a microorganism, preferably a single cell microorganism, comprising preferably native protein, and comprising a gel forming capacity with about 1% to 10% of the protein preparation per total weight of a solution consisting of the protein preparation and water after heat treatment, preferably without syneresis, and optionally:
   a) at least about 70% (w/w), preferably at least about 75% (w/w), more preferably at least about 85% (w/w) and most preferably at least about 95% (w/w) of protein by dry weight of the protein preparation,
   b) about 110 mg/g or less, preferably about 50 mg/g or less, more preferably about 40 mg/g or less, even more preferably about 20 mg/g or less, even more preferably 15 mg/g of lipid by dry weight of the protein preparation,
   c) a water binding capacity of about 4 g/g or more, preferably about 5 g/g or more, more preferably 6.5 g/g or more by dry weight of the protein preparation after heat treatment,
   d) an oil binding capacity of about 0.3 g/g or more, preferably about 0.5 g/g or more, about 2 g/g or more, more preferably about 3 g/g or more, or about 0.3 to 4 g/g, about 0.5 g/g to 4 g/g, about 0.5 g/g to 3 g/g, about 0.5 g/g to 2 g/g or about 0.5 to 0.7 g/g by dry weight of the protein preparation after heat treatment,
   e) a powder solubility of about 74% or more, preferably about 80% of the initial concentration of the protein preparation suspended in water by total weight of the suspension,
   f) a gel forming capacity with about 2%, about 3%, about 4%, about 5% or about 5.5% of the protein preparation per total weight of a suspension consisting of protein preparation and water after heat treatment, preferably without syneresis,
   g) an emulsion activity of about 54% or more, preferably about 55% or more and more preferably 56% or more per total volume of an emulsion consisting of 25 ml sunflower oil and 25 ml solution consisting of 5% protein preparation powder solution in water (w/v) after centrifugation, wherein the emulsion activity is defined as emulsion layer (ml)/total volume (ml)×100%,
   h) an emulsion stability of about 97% or more, preferably about 98% or more, more preferably about 100% or more per total volume of an emulsion consisting of 25 ml sunflower oil and 25 ml solution consisting of 5% protein preparation powder solution in water (w/v) after subjecting the emulsion to 80° C. for 30 min. and subsequent centrifugation, wherein the emulsion stability is defined as emulsion layer (ml)/total volume (ml)×100%,
   i) a foaming capacity of about 40% or more, preferably about 48% or more and more preferably about 50% or more of foam volume per total volume of a dispersion consisting of 10 ml water in 100 mg protein preparation, wherein foam capacity is defined as foam volume (ml)/total volume (ml)×100%,
   j) a foaming stability of about 20% or more, preferably about 40% or more and more preferably about 75% or more of foam volume per total volume of a dispersion consisting of 10 ml water in 100 mg protein preparation after 60 minutes, wherein foam stability is defined as foam volume (ml) measured after 60 minutes/initial volume (ml)×100%, and/or
   k) about 10% (w/w) or less, more preferably about 5.5% (w/w) or less, even more preferably about 2.5% (w/w) or less nucleic acid by dry weight of the protein preparation.
26. The protein preparation of item 25 wherein the protein preparation is in dry form, preferably in the form of a powder.
27. A method for preparing a protein gel comprising:
   (a) providing the protein preparation according to any one of items 23 to 26,
   (b) mixing the protein preparation with an aqueous carrier fluid, and
   (c) heating the mixture to a temperature of at least about 55° C. to provide the protein gel.
28. Use of the protein preparation of any one of items 23 to 26 for preparing a food product, preferably for human or animal use, or a dietary supplement.
29. The use of the protein preparation of item 28 which acts as equivalent or substitute of methylcellulose.
30. The use of the protein preparation of item 28 or 29 which acts as equivalent or substitute of plant protein, egg protein, preferably an egg yolk protein and/or an egg white protein, meat protein, gluten protein, fish protein and/or a milk protein.
31. The use of the protein preparation of any of one of items 28 to 30 which acts as gelling agent, foaming agent, texturing agent, binding agent, thickening agent, stabilizing agent and/or emulsifying agent.
32. The use of any one of items 28 to 31 wherein the food product or dietary supplement is vegan or a non-animal-derived substitute.
33. Use of the protein preparation of any of items 23 to 26 for providing a food product and/or a dietary supplement without rancid tase.
34. A dietary supplement comprising the protein preparation of any one of items 23 to 26.
35. A food product comprising the protein preparation of any one of items 23 to 26 or the dietary supplement according to item 34.
36. The food product of item 35 wherein the food product is a meat substitute, an egg substitute, fish substitute or a dairy product substitute, preferably a non-animal derived substitute.
37. The food product of item 35 or 36 wherein the food product is a patty, a nugget, a steak, bakery product, dough product, spoonable product, cereal product, dairy product, sausage product, fish product or a minced meat-like product.
38. The dietary supplement product of item 34 wherein said dietary supplement is in the form of a tablet, pill, powder, granulate, or flake.
39. A method of preparing a food product or dietary supplement product without a rancid taste comprising:
   a) providing a protein preparation according to any one of items 23 to 26,
   b) optionally mixing the protein preparation with one or more of further ingredients of said product or supplement, and
   c) preparing the food product or dietary supplement.
40. A method of preparing a food product comprising the use of the protein preparation of any one of item 23-26 wherein the protein preparation is a) a gelling agent, b) a methylcellulose substitute, c) a plant protein substitute, d) meat protein substitute, e) gluten protein substitute, f) milk protein substitute g) fish protein substitute or f) an egg protein substitute, preferably an egg yolk protein substitute and/or an egg white protein substitute.
41. A method of preparing a dietary supplement comprising the use of the protein preparation of any one of items 23-26 wherein the protein preparation is a) a gelling agent or b) a methylcellulose substitute.
42. A method of obtaining native protein of a microorganism comprising:
   a) providing a microorganism, and optionally subjecting the microorganism to one or more pre-treatment step(s),
   b) lysing the microorganism thereby preparing a lysate comprising an aqueous liquid fraction comprising nucleic acid and solved native protein of the microorganism, further comprising a step b1) of clearing the lysate, preferably by centrifugation or filtration,
   c) separating the nucleic acid from the aqueous liquid fraction comprising chromatography, wherein the chromatography is anion exchange chromatography and/or anion mixed-mode chromatography:
      i) adding a nucleic acid adsorbent comprising a positively charged functional group immobilized to a solid support, preferably a free-floating solid support to the aqueous liquid fraction,
      ii) optionally stirring or shaking, and
      iii) separating the nucleic acid bound to the nucleic acid adsorbent immobilized to the solid support, preferably by sedimentation and optionally filtration,
      thereby obtaining an aqueous liquid fraction wherein the aqueous liquid fraction is a nucleic acid reduced aqueous liquid fraction,
   d) filtrating the aqueous liquid fraction thereby obtaining a solution comprising purified solved native protein of the microorganism and an aqueous solvent, preferably water or a saline solution, and
   e) optionally removing at least a part of the aqueous solvent,
   f) optionally sterilizing the solution thereby obtaining a sterilized solution comprising purified solved native protein of the microorganism and an aqueous solvent, and
   g) optionally removing at least a part of the aqueous solvent of the sterilized solution of step f).
43. The method of item 42 wherein the nucleic acid adsorbent comprises a quaternary ammonium compound or a hydroxylapatite compound as functional group.
44. The method of item 42 or 43 wherein the method further comprises a step of separating lipid from the aqueous liquid fraction.
45. The method of any one of items 42 to 44 wherein the method further comprises a step b1) of clearing the lysate by centrifugation.
46. The method of any one of items 1 to 22 wherein step c) further comprises quantifying lipid in the aqueous liquid fraction comprising:
   i) contacting the aqueous liquid fraction with a lipophilic solvent, preferably hexane, thereby obtaining a lipophilic phase,
   ii) measuring the absorption, preferably absorption in the UV wavelength range, of the lipophilic phase,
   iii) comparing the absorption measured for the lipophilic phase with a reference absorption measured for the lipid, and
   iv) quantifying the lipid in the lipophilic phase.

### LIST OF FIGURES

FIG. 1: Reduction in absorbance maxima (A, C) and peak areas (B, D) of hexane-extracted brewer's yeast cell lysates before and after use of the skimming separator at the indicated peaks and peak areas using the original peak settings (A and B) and at the more robust later peak setting (C and D).

FIG. 2: Comparison of water binding capacities [g/g] of (A) proteins from pea, faba bean and sunflower with PD yeast protein of the invention with (columns on the right) or without heat treatment (columns on the left) and (B) proteins from pea, faba bean, sunflower, rice and egg white with two different batches of baker's yeast protein and brewer's yeast protein of the invention with heat treatment.

FIG. 3: Peak positive force (top) and positive area (bottom) of measurements with different protein concentrations in a texture analyzer system.

FIG. 4: Comparison of percentual powder solubilities of different proteins with PD yeast proteins of the invention.

FIG. 5: Comparison of emulsion activities of different proteins with PD yeast proteins of the invention.

FIG. 6: Comparison of foam capacities [%] (columns on the left) and foam stability after 60 min [%] (columns on the right) of different proteins with PD yeast proteins of the invention.

FIG. 7: Comparison of storage stability of protein powder with or without skimming separator treatment evaluated by taste evaluation rated from 1-6. (A) Samples were stored as powders at 23° C. (<75% humidity) protected from light or (B) under accelerated storage conditions at 60° C. for up to 182 days.

FIG. 8: Comparison of lipid content in samples treated with and without skimming separator and/or diafiltration. (A) Fat-soluble components detected in the hexane phase provided as absorption at the indicated wavelength, (B) fat-soluble components detected in the hexane phase provided as peak area, and (C) total fat [g/100 g] are provided. Untreated CF/DF (1st column): clarification centrifugation followed by CF/DF and spray drying (without skimming separating); skimming separator treated CF/DF (2^{nd} column): clarification centrifugation followed skimming separation, CF/DF and spray drying; untreated (3^{rd} column): clarification centrifugation and spray drying (without skimming separation and CF/DF); skimm separator treated (4th column): clarification centrifugation followed skimming separation and spray drying.

FIG. 9: Comparison of lipid content in samples treated with and without skimming separator, including fatty acids. (A) Provided is total fat [g/100 g] separator, (B) fatty acid subgroups as indicated and (C and D) fat-soluble components detected in the hexane phase by absorption (C) and peak area (D).

FIG. 10: Comparison of lipid content in samples treated with and without skimming separator. Provided total fat and fatty acid subgroup reduction [%] determined using gas chromatography.

FIG. 11: Left side muffin with PD protein of the invention (recipe muffin vegan), right side conventional muffin.

The invention is further illustrated by the following examples.

### EXAMPLES

### Example 1—Production of a Functional Brewer's Yeast Protein Preparation (Saccharomyces spp.)

Raw material: The yeast biomass (Saccharomyces spp.) was obtained from Kaiser Brauerei GmbH, Geislingen an der Steige, DE.

Hop filtration/sieving: Four liters of brewer's yeast (Saccharomyces carlsbergensis) TS 15% (w/w), pH 5.3 stored in spent yeast, as delivered by the breweries, was sieved with a vibrating sieve or filter bag with a mesh size of 125 μm (120 U.S. Mesh) to remove the residual hop.

Debittering process: The spent yeast was separated from the sieved cell suspension (9-15% w/w) by means of a centrifuge/separator (3000 g, 5 min, 4° C.). The obtained beer-free cell mass was then transferred into a 37° C. warm debittering solution (0.5% Polysorbate 80, 0.2% NaOH, pH 9.1) 1:2 (w/w) and incubated for 10 min (possible range about 10 to about 120 min). The spent debittering solution was then removed by centrifugation and the debittered cell mass was washed with water. The washing process was repeated until the pH of the cell suspension reaches pH of 5.7-6.5 (pH 6.4).

Cell disruption: The cell suspension was adjusted to a dry mass of 12-14% (w/w). The cells were then lysed using a Dyno®-Mill Research Lab of Willy A. Bachofer AG (Muttenz, CH) ball mill (glass beads 0.5 mm, filling quantity 70%, circulation mode: 2.5 L, circulate 45 min; 3.500 rpm) at ° C. 4-8° C. The efficiency of cell disruption was determined by microscopic control (phase contrast method) and protein content (Pierce™ BCA Protein Assay Kit, Thermo Scientific) measured in the supernatant after centrifugation. The protein content for cell disruption degree of 95% was approx. 55 mg ml⁻¹.

Separation of the soluble protein fraction: The lysed cell suspension was centrifuged for 20 min at 17,000 g and 4° C. with Avanti J20 XP Beckman Coulter (Brea, US) to separate the supernatant from the yeast cell walls. The supernatant obtained has the following properties:

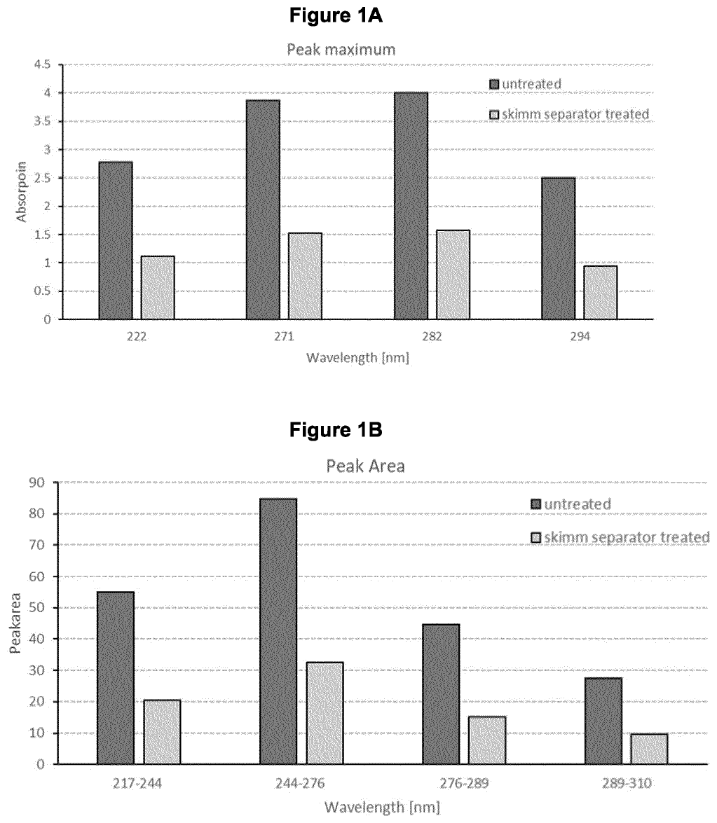

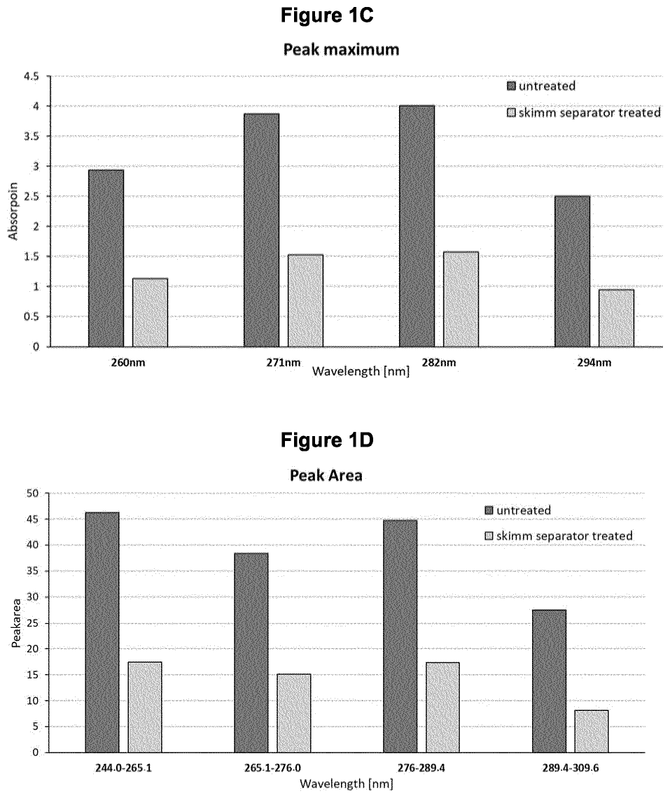

Reduction of the lipid fraction: The soluble protein fraction was centrifuged using a skimming separator (10 500 rpm, bowl diameter 365 mm, disks 12 pcs.) and a feed rate of approx. 1 L/min. The efficiency of lipid separation was determined by UV spectrometry (200-350 nm). The fat-soluble components detected in the hexane phase were reduced by 50-60% (see FIGS. 1A and B). In later examples the measurement of the absorption maxima at 222 nm and the corresponding peak area have been replaced with the absorption maxima at 260 nm (measured peaks: 260 mm, 271 nm, 282 nm and 294 nm; integration peak area: 244-265 nm, 265-276 nm, 276-289 nm and 289-310 nm), as this peak turned out to give more robust results. The results for the same sample measured using the new settings are shown in FIGS. 1C and D. In a separate experiment a reduction of about 20% total lipid (fat) content was observed using the method for determination of total fat content in cereal products after acid digestion by extraction and gravimetry (according to § 64 LFGB L 16.00-5: 2017-10). The designation § 64 LFGB L 16.00-5: 2017-10 describes a method carried out in accordance with DIN standards by a DAkks-certified laboratory (holding an accreditation certificate from the Deutsche Akkreditierungsstelle). The method can be found for example at Beuth Verlag GmbH in the BVL method collection for foods.

Reduction of the nucleic acid content: The chromatography material was added to the 150 ml of lipid reduced supernatant after appropriate pretreatment (equilibration) with a chromatography material concentration of 80 mg ml⁻¹. The supernatant was then incubated with shaking (30 min on a level 6, 40 rpm) in an overhead shaker (STR4 Haberle LABORTECHNIK GmbH+Co. KG). After binding, the chromatography material was separated from the sample by centrifugation (3 345 g, 18° C., 1 min.).

Taste optimization by diafiltration (UF/DF): The lipid-reduced and nucleic acid-reduced supernatant was treated by diafiltration (DF=0.667-2 filtration unit Akta Flux S Cytiva) and a hydrophilic membrane (MWCO 10 kDa, filter area 0.02 m²). H₂O at 12° C. was added as diafiltration buffer. After the diafiltration (DF=0.667-2) was finished, the supernatant was concentrated threefold (CF=3).

TABLE 1
Characteristics of brewer's yeast protein concentrate

| | Characteristic range |
|---|---|
| | of the respective |
| | parameter for the |
| | brewer's yeast |
| Parameter | protein powder |
| Dry matter content | ≥96% |
| Proportion of nucleic acids in the dry matter | 5.5-6.5% |
| Proportion of total lipids in the dry matter* | 3.4-9.3%** |
| Proportion of proteins in dry matter | 75-80% |

*according to §64 LFGB L 16.00-5: 2017 October
**brewer's yeast has a higher lipid content compared to baker's yeast

Drying: The brewer's yeast protein concentrate was dried by means of a spray dryer with an input temperature between 180-155° C., and a resulting output temperature 75° C.-80° C. (Spray Dryer B-290 from Büchi Labortechnik GmbH).

Preservation/sterile filtration: Sterile filtration of a brewer's yeast protein concentrate was carried out in a separate experiment by separating unwanted particles using a heterogeneous PES double membrane+glass fiber membrane (0.8 μm+0.2 μm).

### Example 2—Water Binding Capacity of the Protein Preparation

### Method:

0.5 g sample (powder from Example 1) was mixed with 4 mL demineralized water for 30 s. For samples with higher water absorption the water amount was increased to 5 mL.
Mixture was shaken in a test-tube vibrator for 20 s.
Shaking was repeated seven times in intervals of 10 minutes.
Determination after heat treatment: the mixture was heated to 80° C., held at 80° C. for 10 minutes and let it cool down to room temperature.
Mixture was centrifugated at 20° C., 2 000 g for 25 minutes.
Supernatant was decanted.
For drip off the remaining water: the test tubes were positioned in a 20° angle for 10 minutes.
Calculation

$$WBC = \frac{\begin{matrix}weight of the sample saturated with water - \\ weight of test tube - protein mass\end{matrix}}{protein mass}$$

TABLE 2
Results: comparison without and with heat treatment.

| | without | after heat |
|---|---|---|
| | treatment | treatment |
| Water binding capacity | [g/g] | [g/g] |
| Pea protein 1 | 3.6 | 3.8 |
| Pea protein 2 | 2.3 | 2.5 |
| Faba Bean protein | 0.6 | 2.7 |
| Sunflower protein | 2.4 | 3.0 |
| PD Brewer's Yeast Protein (invention) | 0.1 | 6.9 |

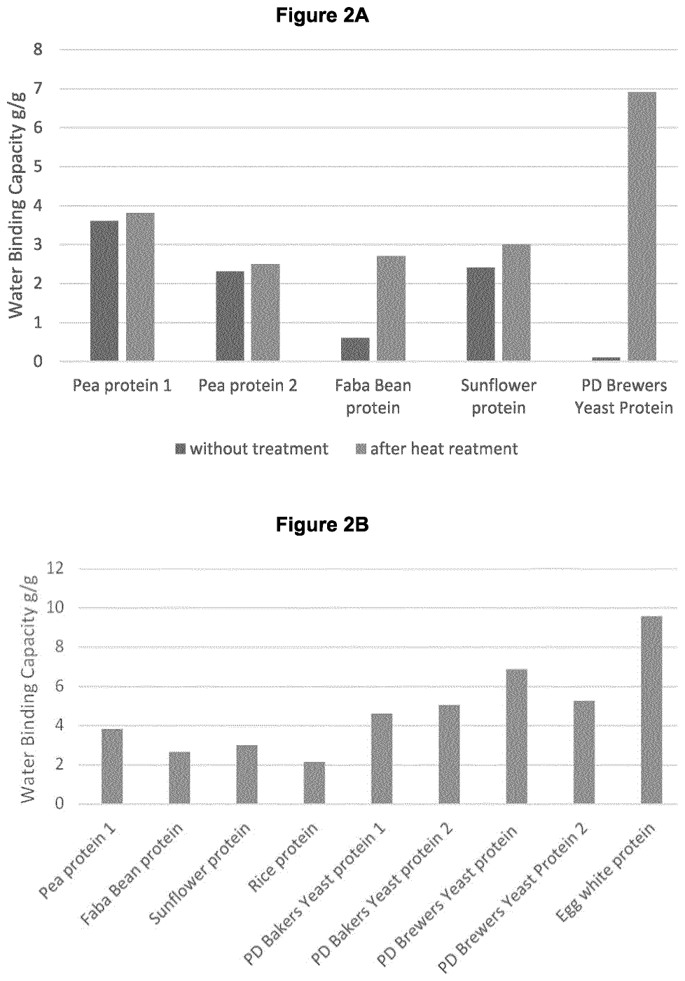

The results are further illustrated in FIG. 2A. It is obvious that based on the high solubility of the PD Yeast protein of the invention without heat treatment no water binding capacity is measurable. After heat treatment a very high water binding capacity of 6-6.9 g/g was found. The method also gives information about the native state of the protein.

TABLE 3
Comparison of the water binding capacity of different proteins with
different inventive proteins (baker's yeast and brewer's yeast)

| Protein | Water Binding Capacity g/g |
|---|---|
| Pea protein 1 | 3.8 |
| Faba Bean protein | 2.7 |
| Sunflower protein | 3.0 |
| Rice protein | 2.2 |
| PD Baker's Yeast protein 1 (invention) | 4.6 |
| PD Baker's Yeast protein 2 (invention) | 5.1 |
| PD Brewer's Yeast protein 1 (invention) | 6.9 |
| PD Brewer's Yeast protein 2 (invention) | 5.3 |
| Egg white protein | 9.6 |

The results are further illustrated in FIG. 2B. Inventive baker's yeast proteins were in a protein concentration range of 50-65%, inventive brewer's yeast proteins in a protein concentration range of 70-80% protein. Plant and egg white proteins were 80% concentrates protein content except for Faba Bean and sunflower protein (60%). It could be proven that water binding capacities for PD proteins of the invention in all cases were above 4.5 g/g. Highest values were detected for brewer's yeast protein with around 7 g/g compared to 2.2 g/g to 3.8 g/g of conventional plant protein. The results confirm that the inventive proteins have an improved water binding capacity compared to conventional plant proteins. Specifically, the values of the inventive proteins are closer to the value of egg white protein with 9.6 g/g confirming that the inventive proteins are particularly useful as egg substitute or equivalent.

### Example 3 Gel Forming Capacity of the Protein Preparation

### Method 1:

Yeast protein (powder of Example 1 Brewer's Yeast protein 1) was dispersed in water,
Dispersion was stirred for 20 min,
Dispersion was spread in 15 ml Sarstedt tubes,
Dispersion was treated for 20 min at 80° C. in a water bath,
Evaluation: fixed: no water loss when turning over tubes, not fixed: water loss.

TABLE 4
Results for PD brewer's yeast protein 1

| | | | Theoretical water binding |
|---|---|---|---|
| Weighing [g] | Water [g] | Gel | [g/g] |
| 0.25 | 3.75 | Solid | 15 |
| 0.25 | 4.375 | Solid | 17.5 |
| 0.25 | 4.75 | Solid | 19 |
| 0.25 | 5 | Solid | 20 |
| 0.25 | 5.625 | Solid | 22.5 |

### Method 2:

A 5% dispersion of protein preparation in water was prepared,
Dispersion was stirred for 20 min,
Dispersion was spread in 15 ml Sarstedt tubes,
Dispersion was treated for 20 min at 80° C. in a water bath,
Evaluation: visual with marks 0-5; 0=bad to 5=excellent.

TABLE 5
Results

| | | Colour | | | |
|---|---|---|---|---|---|
| Protein | Visual solubility | dispersion | Dispersion | Gel | Comment |
| Pea protein 1 | Strong sediment | transparent, | 1 | 0 | Sediment, |
| | | pale brownish, | | | clear |
| | | | | | supernatant, |
| Pea- | Little sediment, | transparent | 3 | 0 | Less |
| protein 2 | foam | pale-yellowish | | | sediment |
| | | | | | Sediment, |
| | | | | | yellowish |
| | | | | | supernatant |
| Egg white | Very good | clear | 5 | 5 | |
| protein | | | | | |
| Potato protein | Very good | clear | 5 | 0 | Clear liquid |
| 20% | | | | | |
| PD Brewer's | Good, no | brownish, bit | 5 | 5 | Greyish gel, |
| Yeast protein 1 | sediment | turbid | | | no syneresis |
| (invention) | | | | | |
| PD Brewer's | Good, no | brownish, not | 5 | 5 | Greyish gel, |
| Yeast protein 1a | sediment | transparent | | | no syneresis |
| (invention) | | | | | |
| PD Brewer's | Good, no | brownish, bit | 5 | 5 | Greyish gel, |
| Yeast protein 1b | sediment | turbid | | | no syneresis |
| (invention) | | | | | |
| PD Baker's | Very good | pale brownish, | 5 | 5 | Greyish gel, |
| Yeast protein 1 | | no foam | | | no syneresis |
| (invention) | | | | | |
| Yeast Protein 1 | Strong sediment, | Light brown | 1 | 0 | Strong |
| (commercial | transparent | | | | sediment, |
| product) | supernatant | | | | transparent |
| | | | | | supernatant |

It was demonstrated that the inventive protein preparations comprising native, functional protein form excellent gels in a protein concentration of 5% after heat treatment which are comparable to the gels obtained with conventional egg white proteins at same concentrations. In contrast, a yeast protein preparation (Yeast Protein 1 in Table 5, Proteissimo, Lesaffre) with a protein concentration of 80% comprising non-functional protein showed no gel forming capacity.

### Method 3: Texture Analyzer Measurements

Different protein solutions (100 mL) were prepared by dissolving the powder in water, while the solution stirs on stirring plate at room temperature. The formation of foam should be avoided. 6 solutions with a concentration of 2.5%, 5%, 7.5%, 10%, 12.5% and 15% (w/w %) were prepared. After the powder was completely dissolved, the pH was adjusted to pH 7, stirred for 10 minutes and measured/adjusted again. Approx. 30 mL of the protein solution were filled in each tube (height: 3.6 cm, diameter 3.3 cm; volume: 30 mL), so that the tube was filled to the top, avoiding the formation of foam. 4 tubes were filled for each concentration for a triple determination and an additional temperature reference sample. Tubes were placed and completely immersed in a water bath at 90° C. for 15 min, allowed to cool to room temperature and stored in the fridge (4° C.) over night.

The gels were measured via a compression test in a texture analyzer in the tubes with a core temperature of 20° C. The core temperature was measured with a thermometer in the additional temperature reference sample.

For the compression test the texture analyzer (Stable Micro Systems; Texture Analyser Model XT2i HR) was loaded with a 5 kg cell and a probe with a diameter of 1.1 cm (Series no.: SMS P/1KS; area 1 cm\hat{ }2) was used. The measurement was conducted with the following test settings: test speed: 1.00 mm/sec; Post-Test-Speed: 10.00 mm/sec; target mode: Distance; Distance: 14.000 mm; Trigger Type: Button; Stop plot at start position, no temperature detection. Before starting the measurement the probe must be placed carefully on the surface of the gel

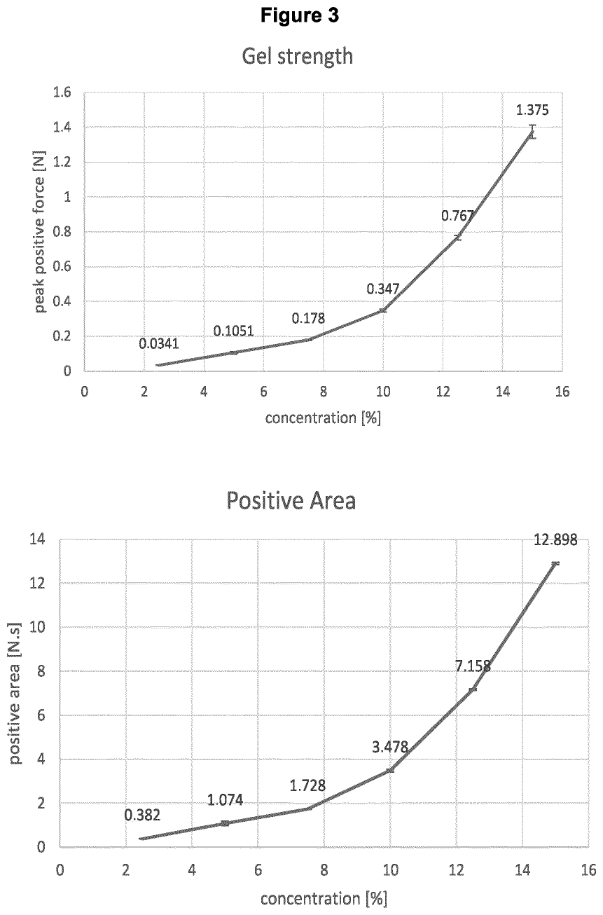

Results: In one batch the gelling strength in dependence of the protein concentration was measured. As shown in FIG. 3 gel strength (peak positive force) is dependent on the protein concentration. Measurable gels were produced with protein concentrations>2%.

Measurement of another batch using above mentioned method at 20° C. showed a gel strength of 0.959 N (standard deviation 0.017N).

### Example 4 Oil Binding Capacity of the Protein Preparation

The same method as in Example 2 was used except that the demineralized water for preparing the 5% protein preparation (powder of Example 1 PD Brewer's yeast protein 2) was replaced by sunflower oil (other plant oils such as rapeseed can also be used) and the oil binding capacity was calculated by (weight of the sample saturated with oil−weight of the test tube−protein mass)/protein mass. The oil binding capacity of the inventive protein was found between 0.5 and 0.7 g/g.

### Example 5 Powder Solubility of the Protein Preparation

### Method:

Source: United States patent, U.S. Pat. No. 4,465,702 (Eastman et al.):
A solution was prepared by adding protein (2%) to 50 mL of demineralized water to a 100 mL beaker,
The solution was stirred on a magnetic stirrer (800 rpm) for 60 minutes,
Samples were transferred to a 50 mL centrifuge tube,
Samples were centrifuged at 2000 g and 20° C. for 25 minutes,
25 mL of the supernatant was transferred into an aluminum shell,
Samples were placed in the drying oven for 1 h 40 min at 160° C.,
Aluminum shells were allowed to cool in the desiccator,
Aluminum shells with dried contents were weighted,
Calculation

$$Solubility [ \% ] = \frac{mass of the sample with alu shell - empty weight of the alu shell}{weight of the suspension \times concentration of protein preparation} \times 2 \times 100 \%$$

TABLE 6
Results for powder solubility is shown

| Protein | Solubility [%] |
|---|---|
| Pea protein 1 | 12.5043 |
| Faba Bean protein | 64.2031 |
| Sunflower protein | 19.8403 |
| Potato protein | 93.0717 |
| PD Brewer's Yeast protein 1 | 75.8040 |
| (invention) | |
| PD Brewer's Yeast protein 2 | 78.8073 |
| (invention) | |
| PD Baker's Yeast protein | 82.1022 |
| (invention) | |
| Egg white protein | 87.1002 |

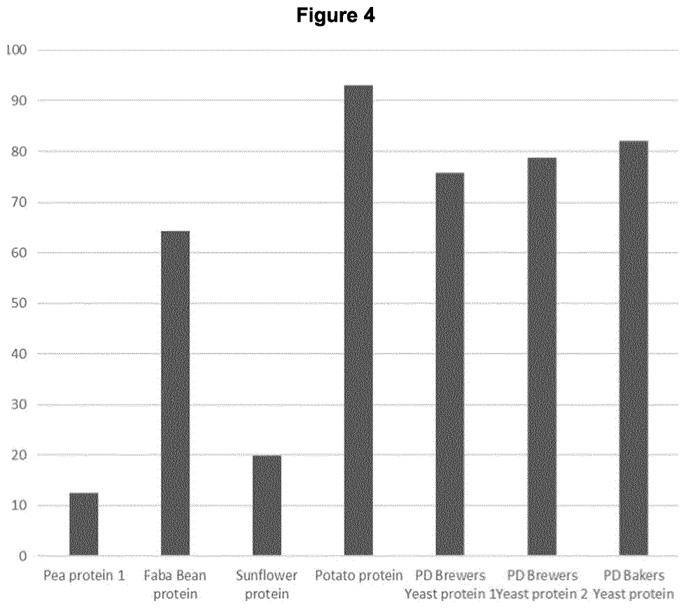

FIG. 4 shows a comparison of percentual powder solubilities of different proteins in comparison with PD yeast proteins of the invention. Baker's yeast proteins of the invention were in a protein concentration range of 50-65%, brewer's yeast proteins of the invention in a protein concentration range of 70-80%. Plant proteins were 80% protein concentrates except for faba bean and sunflower protein (60%) and potato protein (20%). It could be proven that solubilities for PD proteins of the invention in all cases were above 75% relative to the initial amount of protein used in the sample. Highest values for the inventive proteins were detected for baker's yeast protein with around 82%. Egg white protein had a powder solubility of 87.1%.

### Example 6 Emulsion Properties of the Protein Preparation

Emulsion properties were determined with turbidity measurement according to Elif Ezgi Ozdemir, Ahmet Gorgüç, Esra Gençdag, Fatih Mehmet Yilmaz, “Physicochemical, functional and emulsifying properties of plant protein powder from industrial sesame processing waste as affected by spray and freeze drying”, LWT Food Science and Technology, 154 (2022) 112646.

### Method 1: Emulsion Activity

25 ml of sunflower oil was added to 25 ml of a 5% protein powder solution in water,
Homogenization with an IKA T18 at 11000 rpm for 30 s.,
The emulsion was then centrifuged immediately at 1200 g 5 min.,
The amount of the emulsion layer and the total volume were recorded,

$$Emulsion activity ( EA ) [ \% ] = emulsion layer ( mL ) / total volume ( mL ) \times 100$$

### Method 2: Emulsion Stability

25 ml of sunflower oil was added to 25 ml of a 5% protein powder solution in water,
Homogenization with an IKA T18 at 11000 rpm for 30 s.,
The emulsion was then centrifuged immediately at 1200 g 5 min.,
Emulsions were kept for 30 min. in a water bath at 80° C. and cooled quickly,
Samples were centrifuged for 5 min at 1200 g,

$$Emulsion stability ( ES ) [ \% ] = remaining emulsion layer ( ml ) / total \\ \\ volume ( ml ) \times 100.$$

TABLE 7
Results

| | Emulsion activity | Emulsion stability |
|---|---|---|
| Protein | [%] | [%] |
| Pea protein 1 | 53.33 | 96.72 |
| Faba Bean Protein | 52.97 | 100.00 |
| Sunflower protein | 58.04 | 99.40 |
| Potato protein | 53.38 | 96.90 |
| Rice protein | 49.47 | 96.52 |
| PD Brewer's Yeast protein 1 | 56.90 | 100.00 |
| (invention) | | |
| PD Brewer's Yeast protein 2 | 56.09 | 100.00 |
| (invention) | | |
| PD Baker's Yeast protein | 56.39 | 100.00 |
| (invention) | | |

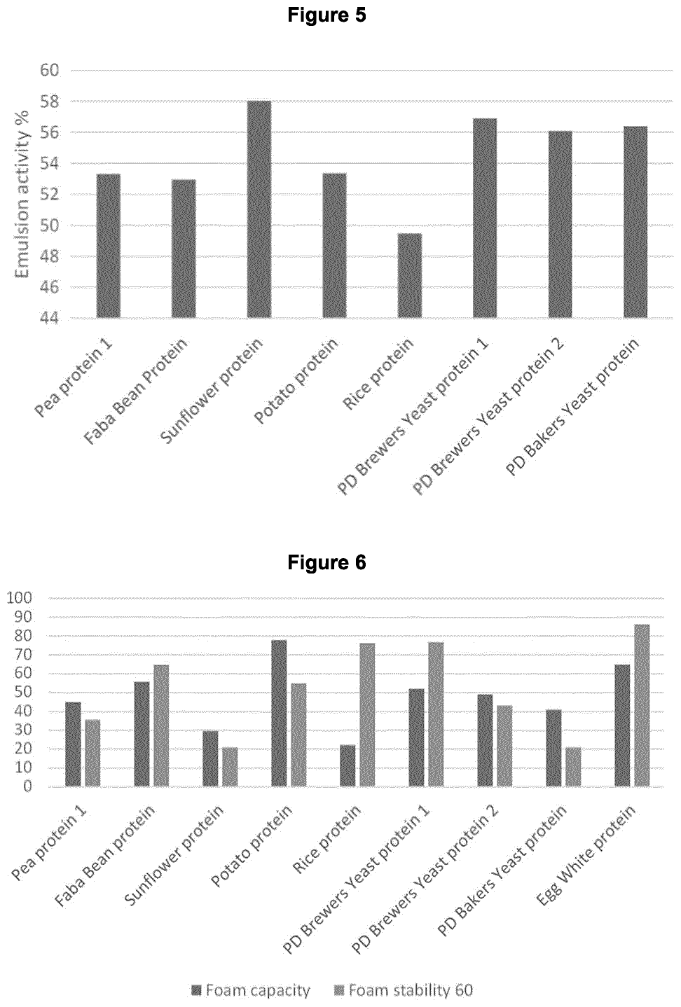

The results are illustrated in FIG. 5 and show a comparison of emulsion activities of different proteins in comparison with PD yeast proteins of the invention. Baker's yeast proteins of the invention were in a concentration range of 50-65%, brewer's yeast proteins of the invention in a range of 70-80% protein. Plant proteins were 80% concentrates protein content except for Faba Bean and sunflower protein in (60%) and potato protein (20%). It could be proven that emulsion activities for PD proteins of the invention in all cases were above 56%. Emulsion stabilities in this test for PD proteins of the invention were 100%.

### Example 7 Foaming Properties of the Protein Preparation

Foaming properties were determined with turbidity measurement according to Elif Ezgi Ozdemir, Ahmet Gorgüç, Esra Gençdag, Fatih Mehmet Yilmaz, “Physicochemical, functional and emulsifying properties of plant protein powder from industrial sesame processing waste as affected by spray and freeze drying”, LWT Food Science and Technology, 154 (2022) 112646.

### Method:

20 ml of distilled water was added to 200 mg of protein powder,
Homogenization e.g., with IKA T18 at 11,000 rpm for 30 s.,
Foam was transferred to a measuring cylinder,
Foam volume after 30 s was documented,

$$Foaming capacity [ \% ] was calculated as the foam volume ( ml ) / total volume of the mixture \times 100.$$

To determine the foam stability, the foam stood for another 60 minutes,
Foam volume was determined after 60 minutes. Foam stability [%] was calculated as foam volume after 60 min/initial foam volume.

TABLE 8
Results

| | Foam capacity | Foam stability 60 |
|---|---|---|
| Protein | [%] | [%] |
| Pea protein 1 | 44.96 | 35.71 |
| Faba Bean protein | 55.74 | 64.80 |
| Sunflower protein | 29.57 | 20.80 |
| Potato protein | 77.83 | 54.95 |
| Rice protein | 22.10 | 76.25 |
| PD Brewer's Yeast protein 1 | 52.08 | 76.77 |
| (invention) | | |
| PD Brewer's Yeast protein 2 | 49.07 | 43.36 |
| (invention) | | |
| PD Baker's Yeast protein | 41.10 | 20.76 |
| (invention) | | |
| Egg White protein | 64.88 | 86.24 |

The results are also shown in FIG. 6. It could be shown that PD proteins of the invention from different sources can be adjusted in a wide spectrum with regard to their foam properties. In particular, the foam stability can be adjusted.

### Example 8 Effects of Lipid Reduction on Storage Stability of Protein Preparations

The storage stability of protein powders can be compromised by factors such as lipid oxidation, which can lead to changes in taste and impaired functionality. One possible method for improving the long-term stability of protein powders is the reduction of lipids contained within.

Two different powders were produced essentially as described in Example 1 with small modifications, wherein one part of the protein solution was treated with a skimming separator (10 500 rpm, bowl diameter 365 mm, disk 12 pcs) to reduce the lipid content and the other part remained untreated.

In brief, twenty litters from brewery TS 15% (w/w), pH 5.3 stored as spent yeast, and delivered by the breweries, was sieved with a vibrating sieve or filter bag with a mesh size of 125 μm to 50 μm (120 U.S. Mesh to 270 U.S. Mesh) to remove the residual hop.

The spent yeast was adjusted to a dry mass between 7-15% w/w and applied to the debittering process essentially as described in Example 1 with small modifications.

The cell suspension was adjusted to a dry mass of 12-14% (w/w). The cells were then lysed using a LabStar Discus Mill NETZSCH-Feinmahltechnik GmbH (filling quantity 70%, passage mode). The efficiency of cell disruption was determined by microscopic control (phase contrast method) and protein content (Pierce™ BCA Protein Assay Kit, Thermo Scientific) measured in the supernatant after centrifugation. The protein content for cell disruption degree of 95% was approx. 55 mg ml⁻¹.

The lysed cell suspension was centrifuged for 20 min at 17,000 g and 4° C. with Avanti J20 XP Beckman Coulter (Brea, US) to separate the supernatant from the yeast cell walls.

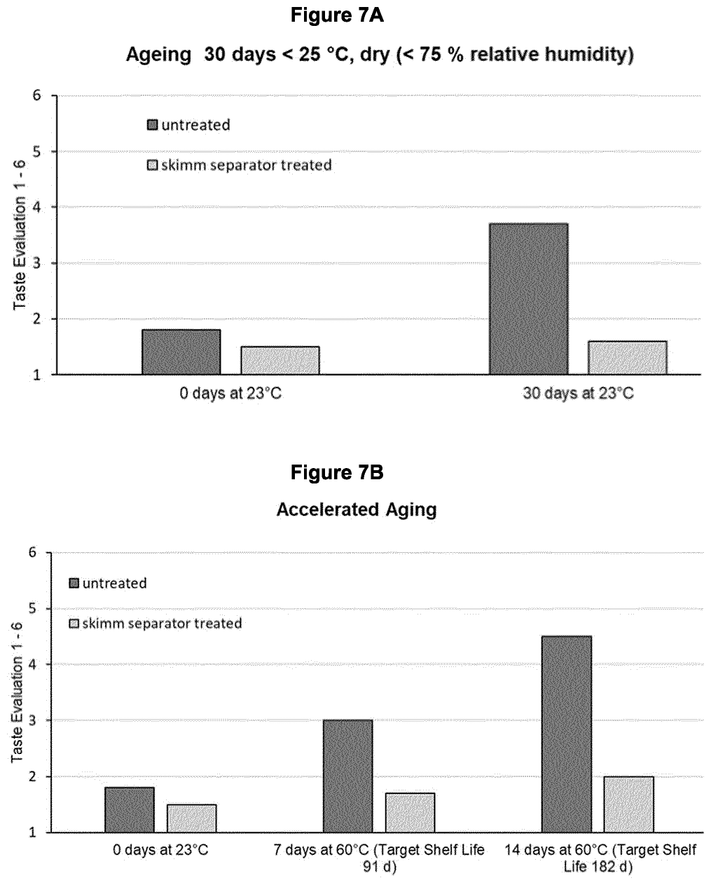

The supernatant obtained was split into two parts. One part of the supernatant was treated with a skimming separator (FIG. 7=skimming separator treated (10 500 rpm, bowl diameter 365 mm, disks 12 pcs., feed rate of approx. 1 L/min) after centrifugation and thus subjected to lipid reduction (skim separator treated), while the other part was processed directly after centrifugation without lipid reduction (FIG. 7=untreated).

Taste optimization by diafiltration (UF/DF): Both untreated and skimming separator treated samples were subjected to diafiltration (DF=0.667-2 SW18 HFC filtration unit—MMS AG) using a hydrophilic membrane (MWCO 10 kDa) and H₂O as diafiltration buffer at 12° C. After the diafiltration (DF=0.667-2) was finished, the supernatant was concentrated threefold (CF=3).

Drying: Both untreated and skimming separator treated and concentrated samples were dried by means of a spray dryer with an input temperature between 180-155° C., and a resulting output temperature 75° C.-80° C. (Spray Dryer B-290 from Bchi Labortechnik GmbH).

The two different powders (skimming separator treated and untreated)d were stored under defined storage conditions for a duration of 1 month, which included a temperature of 23° C., a relative humidity of less than 75%, and light protection (see FIG. 7A). Additionally, accelerated storage tests were performed to evaluate the long-term stability of the two different powder samples (skimming separator treated sample and untreated sample). Two target shelf lives of 91 and 182 days were chosen, and a temperature of 60° C. (TAA=accelerated aging temperature) with a Q10 of 2 was applied, while real-time ambient conditions (TRT) were maintained at 23° C. (FIG. 7B). The acceleration of aging was calculated by determining the accelerated aging time (AAT) using the formula AAT=desired real-time (RT) Q10 [(TAA-TRT)/10].

Finally, the sensory properties of the different powder samples during storage were evaluated by a group of five independent individuals in a blind study. The individuals rated their assessment of the taste changes in the powder samples on a scale of 1 (very good, no rancidity) to 6 (very bad, very rancid).

The data show that lipid reduction using skimming separation significantly improves long-term stability of protein powders resulting in strongly reduced taste changes during storage

### Example 9—Importance of Skim Separation in Protein Solution Processing

As a part of Example 8 the following Experiments were performed additionally:

After lysing the yeast cell suspension, the suspension was centrifuged for 20 min at 17,000 g and 4° C. with Avanti J20 XP Beckman Coulter (Brea, US) to separate the supernatant from the yeast cell walls. The supernatant obtained was split into four parts. Two parts of the supernatant were treated with a skimming separator 10 500 rpm, drum diameter 365 mm, discs 12 pieces, flow rate of approx. 1 L/min) after centrifugation of cell walls and thus subjected to lipid reduction (skimming separator treated), while the other two parts were not subjected to lipid reduction after centrifugation of cell walls.

One sample each of the untreated and skimming separator treated samples was dried to powder without diafiltration (UF/DF) and the other sample was filtered and concentrated with a SW18 HFC UF/DF-System form MMS AG using a hydrophilic membrane (MWCO 10 kDa, filter area 0.16 m2) and H₂O as diafiltration butter at 12° C. (DF=0.667-2, Concentration threefold (CF=3)). These two samples were then dryed as well.

All four powders were analysed by UV spectrometry (200-350 nm), and the total lipid (fat) content was measured by a gravimetric method following Weibull-Stoldt ASU L 06.00-6 (2014-08). Analysis of total lipid (fat) content was performed by external DAkks-certified (holding an accreditation certificate from the Deutsche Akkreditierungsstelle) analytical laboratory. The analysed samples are abbreviated as follows:

untreated: Centrifugation for 20 min at 17,000 g and 4° C. (Avanti J20 XP Beckman Coulter, clarifying step) without skimming separator treatment and dried using spray drying
untreated CF/DF: Centrifugation for 20 min at 17,000 g and 4° C. (Avanti J20 XP Beckman Coulter, clarifying step) without skimming separator treatment, filtered and concentrated with a SW18 HFC UF/DF-System form MMS AG using a hydrophilic membrane (MWCO 10 kDa, filter area 0.16 m2) and dried using a spray dryer.
Skimming separator treated: Centrifugation for 20 min at 17,000 g and 4° C. (Avanti J20 XP Beckman Coulter, clarifying step) with skimming separator treatment and dried using a spray dryer
Skimming separator treated CF/DF: Centrifugation for 20 min at 17,000 g and 4° C. (Avanti J20 XP Beckman Coulter, clarifying step) with Skim separator treatment, filtered and concentrated with a SW18 HFC UF/DF-System form MMS AG using a hydrophilic membrane (MWCO 10 kDa, filter area 0.16 m2) and dried using a spray dryer.

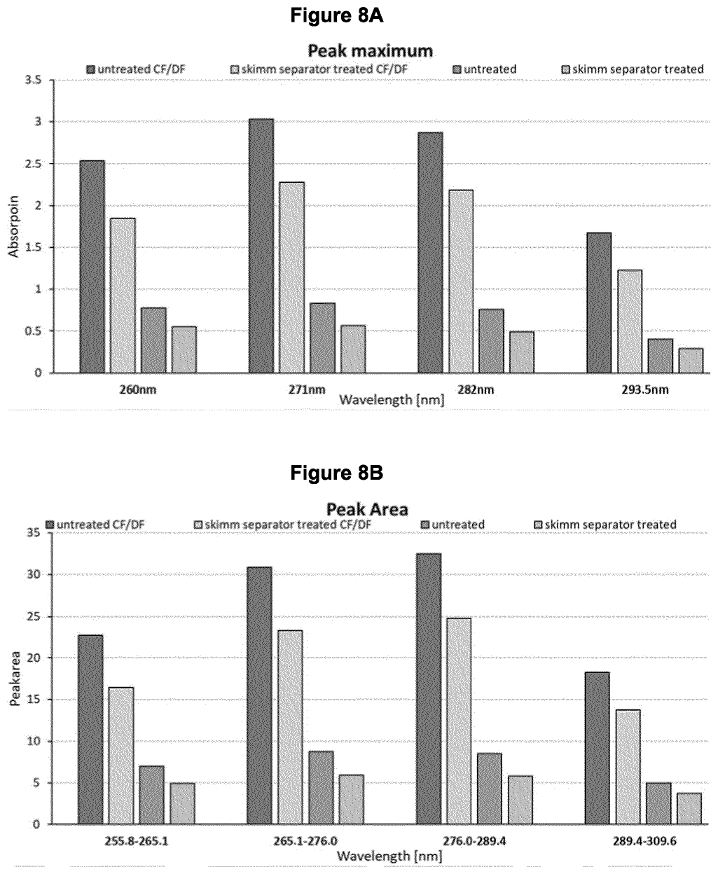

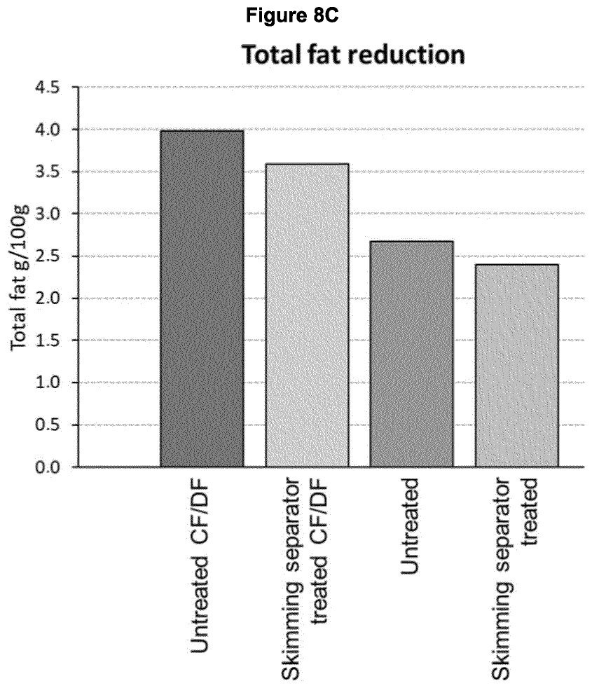

In this example, the fat-soluble components detected in the hexane phase were reduced by 25-30% (FIGS. 8A and B), which corresponds to a total fat reduction of 10-11% (FIG. 8C). During UF-DF/CF, both the fat-soluble components detected in the hexane phase and the total fat reduced by the Skimming separator were concentrated by filtration. Since the reduction profile of the fat-soluble components detected in the hexane phase and the total fat reduction of the unconcentrated samples were similar to the unconcentrated samples (fat-soluble components detected in the hexane phase were reduced by 25-30%, total fat by 10-11%), it is shown that the fat reduction is caused by skimming separation and filtration has no impact on the fat reduction (FIGS. 8A and C). We note in this regard that the detected lipid reduction had a strong influence on taste perception as shown in FIG. 7, particularly following storage of the powder. Moreover, in other experiments lipid reduction has been higher and using an industrial skimming separator, an even higher lipid reduction is expected, as these can be run more accurately.

### Example 10 Lipid Reduction in the Protein Solution Using a Skim Separator (UV Spectometry, Gravimetric Method and Gas Chromatography)

Example 9 was repeated in an independent experiment and samples were taken from the solution treated with a skimming separator to reduce the lipid content, and from the part that remained untreated. The lipid reduction achieved by the skimming separator compared to the untreated solution was determined by UV spectrometry (200-350 nm), and the total lipid (fat) content was measured by a gravimetric method following Weibull-Stoldt ASU L 06.00-6 (2014-08), as well as the content of fatty acids by Gas chromatograph method (ISO 12966-2:2011 mod., GC/FID.)

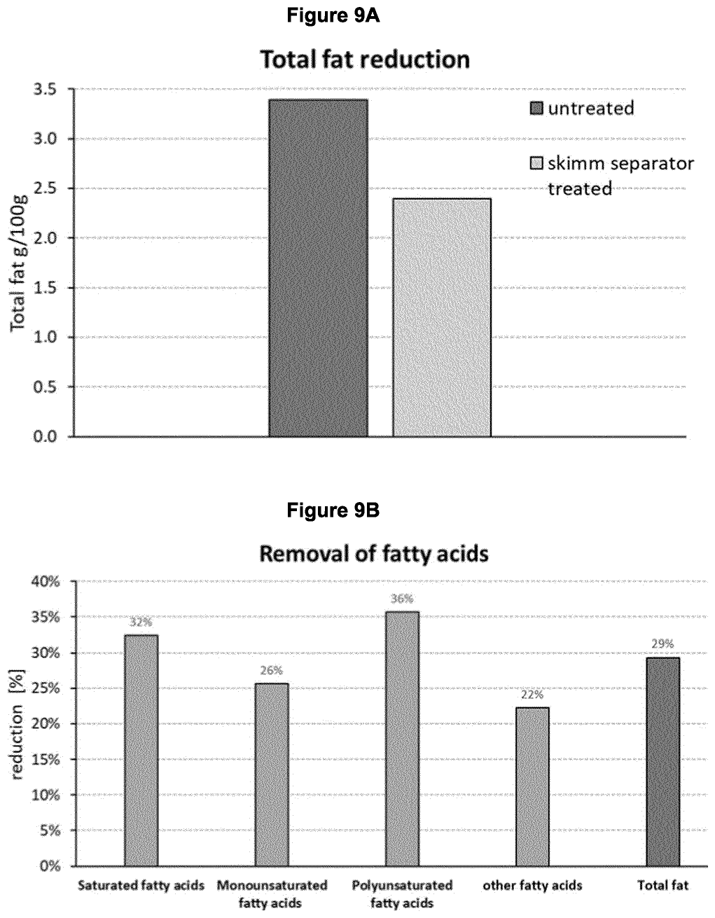

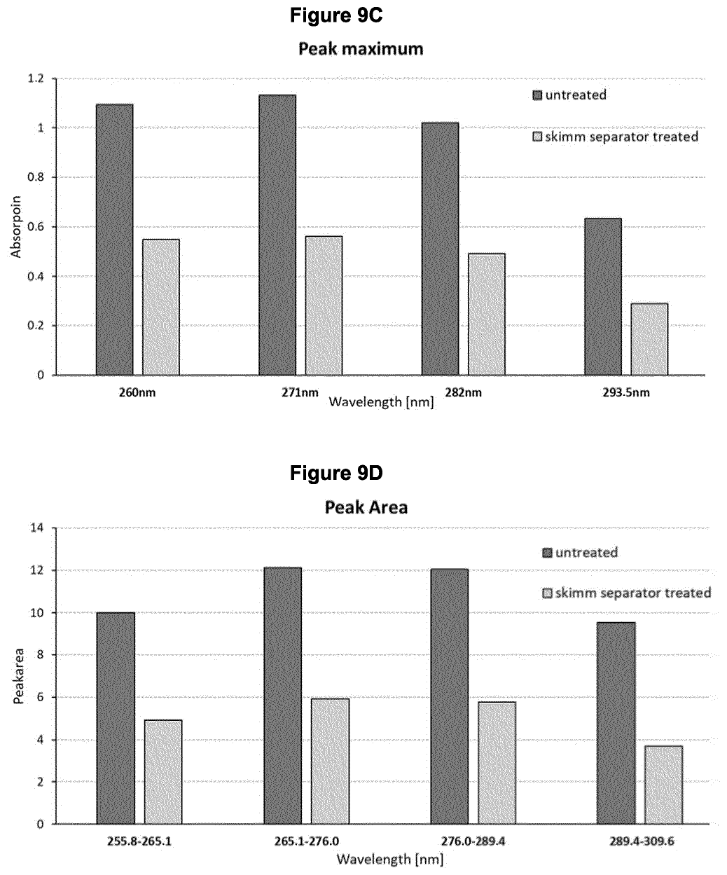

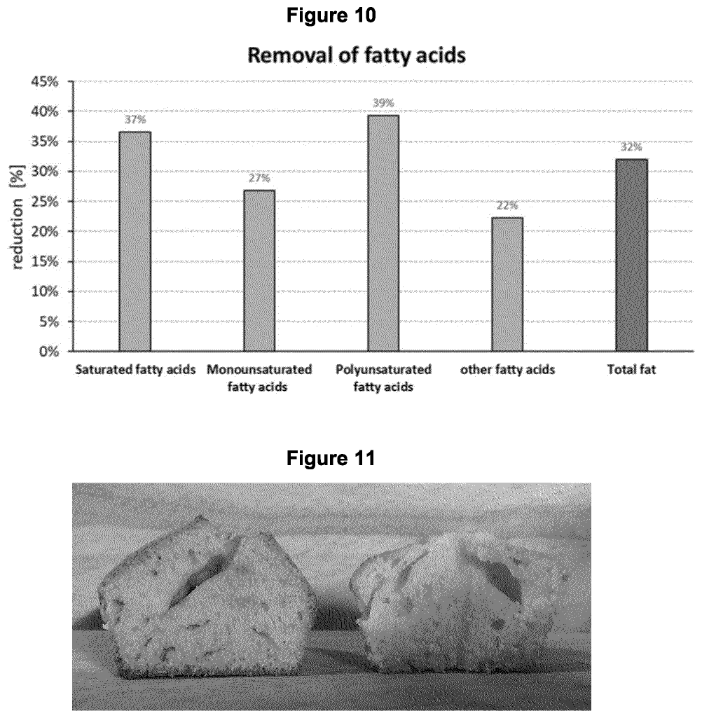

In this example, the fat-soluble components detected in the hexane phase were reduced by 50-55% (FIGS. 9C and D), which corresponds to a total fat reduction of 29% (FIGS. 9A and B) and a specific fatty acid reduction of 22%-36% (FIG. 9B). In another independent experiment, the removal of 32% of the total fat content and a specific fatty acid reduction of 22%-39% (FIG. 10) were demonstrated. Especially the polyunsaturated fatty acids, which tend to oxidize more rapidly and thus become rancid, are reduced significantly by up to 36%-39% by using the skim separator (see FIG. 10), which, as already demonstrated in Experiment 7, improves shelf life and prevents sensory off-flavors (rancidity) of the yeast preparation. In this example, the fat content of the light liquid phase (containing fat), which was removed by the skimming separator, was also analysed. It was found that the total lipids (fat) content in the removed light liquid phase is around 260% higher than in the heavy liquid phase (fat-reduced phase).

### Example 11 Preparation of Muffins with the Protein Preparation

The muffins were prepared according to the following recipe:

TABLE 9

| | Muffin | Muffin vegan | Muffin blind |
|---|---|---|---|
| | conventional | (invention) | experiment |
| Ingedients | [%] | [%] | [%] |
| Butter | 24.3 | | 24.3 |
| Oil/Margarine | 0.0 | 26.0 | 0.0 |
| Flour | 30.0 | 30.0 | 30.0 |
| Sugar | 20.2 | 20.0 | 20.2 |
| Vanilla sugar | 0.3 | 0.3 | 0.3 |
| Salt | 0.1 | 0.1 | 0.1 |
| Eggs | 0.0 | — | 0.0 |
| Egg yolk | 6.1 | — | 0.0 |
| Egg white | 12.1 | — | 0.0 |
| Baking powder | 1.2 | 1.2 | 1.2 |
| PD Brewers Yeast | — | 3.1 | — |
| protein 1 | | | |
| Water | | 13.6 | 18.2 |
| Milk | 5.7 | — | 5.7 |
| Soy milk | — | 5.7 | — |
| | 100.0 | 100.0 | 100.0 |

TABLE 10
Results

| | Muffin | Muffin vegan |
|---|---|---|
| Category | conventional | (invention) |
| Taste** | typical, sweet, pleasant | Less sweet, pleasant |
| Taste** after 7 days | typical, sweet, pleasant | Less sweet, pleasant |
| Ranking** | 2 | 1 |
| Juiciness* | 5 | 5 |
| Juiciness* after 7 days | 5 | 5 |
| Texture fresh | fluffy | Fluffy |
| Texture after 7 days | fluffy | Fluffy |
| Colour**** | yellow | Greyish to brownish* |
| Pore structure | Regular, small | Regular, small |
| Volume mL/100 g | 171.5 | 171.6 |
| batter *** | | |

*1 bad - 5 very good
**5 persons
*** mean value out of 5
****No coloring was used to replace egg yolk.

FIG. 11 shows a comparison between the vegan muffin of the invention (left side) and a conventional muffin (right side). It has been shown that masses can be produced on the basis of a PD protein of the invention that lead to sponge cakes (e.g. muffins) that are comparable to or better than the variants made with egg in terms of taste, juiciness, texture (including storage stability), pore structure and volume.

### Example 12 Preparation of Angel Cake with the Protein Preparation

Angel cake is one of the best known food models for testing food protein foaming and gelation simultaneously. Cake height, texture, and compressibility appear to be related to four elementary characteristics; viscosity, foaming capacity (FC), foaming stability (FS) and gelation (Kneifel, W. and Seiler, A. (1993) “Water-holding Properties of Milk Protein Products—A Review,” Food Structure: Vol. 12: No. 3, Article 3).

The angel cake was prepared according to the following recipe:

TABLE 11

| | | Vegan |
|---|---|---|
| | conventional | (invention) |
| Ingredients | [%] | [%] |
| Flour | 15.7 | 15.7 |
| Sugar | 36.6 | 36.6 |
| Aroma (Vanilla sugar) | 0.9 | 0.9 |
| Salt | 0.2 | 0.2 |
| Baking powder | 0.9 | 0.9 |
| Egg white | 45.7 | |
| PD Protein | 0.0 | 5.0 |
| (invention) | | |
| Water | 0.0 | 40.7 |
| | 100.0 | 100.0 |

Recipe adapted from Kneifel et al.

Angel Cake manufacturing: protein dispersion in water or egg white was whipped to form a thick foam. Sucrose was added. Then flour was added to produce the cake batter which was baked at 88° C. for 30 min. It could be shown that the proteins of the invention (PD Protein) can substitute egg.

### Example 13 Preparation of Scrambled Egg with the Protein Preparation

The scrambled egg was prepared according to the following recipe:

TABLE 12

| | Ingredient [%] |
|---|---|
| PD Brewer's Yeast Protein 1 | 5 |
| (invention) | |
| Locust Bean Gum (LBG)* | 1 |
| Carageenan* | 0-1.5 |
| Starch | 1 |
| Yeast Flakes | 5 |
| Rapeseed oil | 15 |
| Water | 67.5 |
| Salt with herbs | 0.5 |
| Kala Namak | 1 |
| White pepper | 0.5 |
| Mustard | 1 |
| Tomato paste | 0.5 |
| Kurkuma | 0.5 |

*Can be made without carageenan and also with other thickeners (e.g., citrus fiber).

Dry ingredients were mixed, mustard and tomato paste were added to water,
Mass was mixed in the blender and oil was gradually added,
Mass was fried in a pan with oil, set and a silicone scraper was used to break up the scrambled eggs

### Result:

Scrambled egg of the invention has a consistency similar to scrambled egg using egg. Thus, the protein preparation of the invention is suitable as egg substitute.

### Example 14 Preparation of Burger Patties with the Protein Preparation

Method 1: The following recipe has been used:

TABLE 13

| | PD1 | PD2 | PD3 | PD4 | E1 | EMC |
|---|---|---|---|---|---|---|
| Ingredients | [%] | [%] | [%] | [%] | [%] | [%] |
| Oil | 10 | 10 | 10 | 10 | 10 | 10 |
| Water | 56 | 49 | 49 | 50 | 56 | 55 |
| PD Brewer's Yeast | 8 | 15 | 15 | 12 | 0 | 0 |
| protein 1 (invention) | | | | | | |
| Pea protein 1 | — | — | — | — | 8 | 8 |
| Pea protein texturatet | 10 | 10 | 10 | 10 | 10 | 10 |
| Xanthane | 1 | 1 | 0 | 0 | 1 | 1 |
| Salt | 1 | 1 | 1 | 1 | 1 | 1 |
| Starch | 4.5 | 4.5 | 5.5 | 5 | 4.5 | 4.5 |
| Flour | 8 | 8 | 8 | 8 | 8 | 8 |
| Vinegar | 0.5 | 0.5 | 0.5 | 0.5 | 0.5 | 0.5 |
| Methylcellulose | — | — | — | — | — | 1 |
| Spices | 1 | 1 | 1 | 1 | 1 | 1 |
| | 100 | 100 | 100 | 100 | 100 | 100 |
| Results | soft | Pattie solid + | Pattie solid + | Very soft | Pattie | Pattie solid, |
| | | minced-meat-like. | minced-meat-like. | | disintegrates, | mass formable |
| | | solid. formable | solid. formable. | | mass formable | |
| | | mass | a bit sticky mass | | | |

### Preparation:

Starch was dispersed with 25% of the water and heated,
Texture was soaked with 50% of the water for at least 1 hour,
Protein was dispersed and, if necessary, hydrocolloids with another 25% of the water,
For methyl cellulose (EMC): methyl cellulose and oil and a small portion of the water was dispersed followed by adding the protein dispersion, mass was cooled (overnight at 4° C.),
The mass was formed into a patty and fried

### Method 2: Own Recipe

TABLE 14

| Ingredients | % |
|---|---|
| PD Protein | 12.5 |
| Texturate | 10 |
| Starch | 5 |
| Flour | 5 |
| Rapeseed oil | 10 |
| Water | 50 |
| Vinegar | 0.5 |
| Yeast Flakes | 1.5 |
| Salt with herbs | 1.5 |
| White pepper | 0.5 |
| Smoked paprica powder | 1.5 |
| Garlic powder | 1 |
| Tomato paste | 1 |

Preparation was carried out according to the following protocol:
Mix all the “seasoning ingredients” with half the water and soak the textures in it until it is soft (at least 45 minutes)
Dissolve/disperse PD Protein in half of the remaining water
Heat the other half of the remaining water and dissolve the starch in it (Q viscous starch slurry)
Mix together all the components from steps 1-3, as well as the oil and flour, creating a thick paste
Shape and fry in oil over medium heat
   Method 3: Based on recipe of Method 2 different concentrations of inventive yeast protein, pea protein and methyl cellulose were used.

TABLE 15

| | 12.5 | 2 | 3 | 5 | 0 | MC |
|---|---|---|---|---|---|---|
| | % | % | % | % | % | % |
| Pea Protein 1 | 0 | 10.5 | 9.5 | 0 | 12.5 | 10.5 |
| PD Brewers | 12.5 | 2 | 3 | 5 | 0 | 0 |
| Yeast | | | | | | |
| Protein 2 | | | | | | |
| Pea protein | 10 | 10 | 10 | 10 | 10 | 10 |
| texturate | | | | | | |
| Methylcellulose | 0 | 0 | 0 | 0 | 0 | 2 |
| Stability | stable | less stable | stable | stable | disintegrate | stable |
| during frying | | | | | | |
| Elasticity | very good | low | good | good | dry, not | lower |
| | | elasticity | elasticity | elasticity | elastic | elasticity |
| | | | | | | compared |
| | | | | | | to PD |
| Juiciness | juicy | less juicy | juicy | juicy | Dry | juicy |
| Taste | spicy taste | slight | neutral | bit spicy | off taste | off taste |
| | | off taste | taste | taste | (pea flavor) | (pea flavor) |
| | | (pea flavor) | | | | |
| Colour | like fried | brownish | less | less | brownish | brownish |
| | meat, not | colour | brownish | brownish | colour | colour |
| | brownish | | | | | |
| | as with | | | | | |
| | pea protein | | | | | |
| Batter | Semi liquid | formable | formable | formable | formable | formable |
| texture | | | | | | |

### Results:

### Method 1:

The patties with methylcellulose (EMC) held together when fried. Patties with the pea protein-based recipe (E1) fell apart easily when frying. The mass was easy to shape. Patties based on PD 2 and 3 were easy to shape and stable when fried (comparable EMC). The results confirm that the protein of the invention can be used as a substitute for methylcellulose.
Method 2 is an adapted recipe with spices (final application recipe),
Method 3: Tests showed that a concentration of 2-3% in then regarded basic recipe brought an effect comparable or even better than methylcellulose 2%. Inventive yeast protein can be used in smaller concentrations and as substitute of parts of other proteins as pea protein.

### Example 15 Protein Preparation as Substitute for Dairy Products (Examples Dairy Replacer)

TABLE 16
Ingredients

| | % |
|---|---|
| PD Protein | 5 |
| LBG* | 1 |
| Starch | 1 |
| Yeast Flakes | 5 |
| Coconut fat | 15 |
| Water | 69 |
| Salt with herbs | 1.5 |
| White pepper | 0.5 |
| Garlic | 0.5 |
| Mustard | 1 |
| Tomato paste | 0.5 |

*Can also be made with other thickening ingredients instead of LBG (e.g., psyllium or citrus fiber)

Preparation was carried out according to the following protocol:
1. Mix dry ingredients, add mustard and tomato paste to the water
2. Mix the ingredients from step 1, melt fat and add gradually
3. Gently heat in the pot at the smallest level
4. Put in a form and cool down
   Protein content can also be increased.

1. A method of preparing native protein of a microorganism comprising:
a) providing the microorganism, and optionally subjecting the microorganism to one or more pre-treatment step(s),
b) lysing the microorganism thereby obtaining a lysate comprising an aqueous liquid fraction comprising lipid and solved native protein of the microorganism, further comprising a step b1) of clearing the lysate, preferably by centrifugation or filtration,
c) separating the lipid from the aqueous liquid fraction using mechanical means thereby obtaining an aqueous liquid fraction wherein the aqueous liquid fraction is a lipid reduced aqueous liquid fraction comprising the solved native protein of the microorganism, wherein separating the lipid is performed by a centrifugal three-phase separator,
d) filtrating the aqueous liquid fraction thereby obtaining a solution comprising purified solved native protein of the microorganism and an aqueous solvent, preferably water or a saline solution,
e) optionally removing at least a part of the aqueous solvent,
f) optionally sterilizing the solution thereby obtaining a sterilized solution comprising purified solved native protein of the microorganism and an aqueous solvent, and
g) optionally removing at least a part of the aqueous solvent of the sterilized solution.

2. The method of claim 1 wherein separating the lipid from the aqueous liquid fraction using mechanical means in step c) is performed by a skimming separator and/or a three-phase decanter.

3. The method of claim 1 wherein filtrating in step d) is ultrafiltration, preferably diafiltration/ultrafiltration, more preferably with a molecular weight cut-off in a range of about 1 kDa to about 100 kDa, preferably of about 3 kDa to about 50 kDa, more preferably of about 5 kDa to about 15 kDa, most preferably of about 10 kDa.

4. The method of claim 1, wherein the microorganism is a eukaryotic microorganism, preferably a eukaryotic microorganism selected from the group consisting of a fungus, preferably Aspergillus niger; a yeast, preferably selected from the group consisting of Saccharomyces spp., preferably S. cerevisiae, S. carlsbergensis, S. bayanus, S. ellipsoides, S. uvarum, S. ludwigii or S. pastorianus; Pichia spp., preferably P. pastoris; Hansenula spp.; Candida spp., preferably C. utilis; Torulopsis spp.; and Yarrowia lipolytica; and an alga, preferably Arthrospira maxima (Spirulina maxima), Arthospira platensis (Spirulina platensis), Chlorella vulgaris or Euglena gracilis.

5. The method of claim 1, wherein the microorganism is a prokaryotic microorganism, particularly a bacterium selected from the group consisting of Bacillus subtilis, Lactobacillus spp., Corynebacterium glutamicum, Methylomonas spp., Spirulina ssp., and Xanthomonas spp.

6. The method of claim 1, wherein the method comprises a step of clearing the lysate in step b1) by centrifugation.

7. The method of claim 1, wherein steps b) to g), preferably steps b) to d), are performed at a temperature of about 40° C. or less, preferably at a temperature in the range of about 30° C. to 20° C.

8. The method of claim 1, wherein the method comprises a further step of separating nucleic acid from the aqueous liquid fraction, preferably from the lipid reduced aqueous liquid fraction comprising the solved native protein of the microorganism of step c), or the solution, wherein the step of separating nucleic acid comprises anion-exchange chromatography and/or anion mixed-mode chromatography.

9. A protein preparation obtainable by the method according to claim 1, wherein preferably the protein preparation comprises a gel forming capacity with about 1% to 10% of the protein preparation per total weight of a solution consisting of the protein preparation and water after heat treatment, preferably without syneresis, and optionally
a) at least about 70% (w/w), preferably at least about 75% (w/w), more preferably at least about 85% (w/w) and most preferably at least about 95% (w/w) of protein by dry weight of the protein preparation,
b) about 110 mg/g or less, preferably about 50 mg/g or less, more preferably about 40 mg/g or less, even more preferably about 20 mg/g or less, even more preferably about 15 mg/g or less of lipid by dry weight of the protein preparation,
c) a water binding capacity after heat treatment of about 4 g/g or more, preferably about 5 g/g or more, more preferably 6.5 g/g or more by dry weight of the protein preparation,
d) a gel forming capacity with about 2%, about 3%, about 5% or about 5.5% of the protein preparation per total weight of a solution consisting of the protein preparation and water after heat treatment, preferably without syneresis, and/or
e) about 10% (w/w) or less, more preferably about 5.5% (w/w) or less, more preferably about 2.5% (w/w) or less nucleic acid by dry weight of the protein preparation.

10. A protein preparation derived from a microorganism, preferably a single cell microorganism, comprising a gel forming capacity with about 1% to 10% of the protein preparation per total weight of a solution consisting of the protein preparation and water after heat treatment, preferably without syneresis and optionally:
a) at least about 70% (w/w), preferably at least about 75% (w/w), more preferably at least about 85% (w/w) and most preferably at least about 95% (w/w) of protein by dry weight of the protein preparation,
b) about 110 mg/g or less, preferably about 50 mg/g or less, more preferably about 40 mg/g or less, even more preferably about 20 mg/g or less, even more preferably about 15 mg/g or less of lipid by dry weight of the protein preparation,
c) a water binding capacity of about 4 g/g or more, preferably about 5 g/g or more, more preferably 6.5 g/g or more by dry weight of the protein preparation after heat treatment,
d) a gel forming capacity with about 2%, about 3%, or about 5% or about 5.5% of the protein preparation per total weight of a solution consisting of the protein preparation and water after heat treatment, preferably without syneresis, and/or
e) about 10% (w/w) or less, more preferably about 5.5% (w/w) or less more preferably about 2.5% (w/w) or less nucleic acid by dry weight of the protein preparation.

11. The protein preparation of claim 9 wherein the protein preparation is in dry form, preferably in the form of a powder.

12. A method for preparing a protein gel comprising:
(a) providing the protein preparation according to claim 9,
(b) mixing the protein preparation with an aqueous carrier fluid, and
(c) heating the mixture to a temperature of at least about 55° C. to provide the protein gel.

13. (canceled)

14. A dietary supplement or a food product comprising the protein preparation of claim 9.

15. A method of preparing native protein of a microorganism comprising:
a) providing a microorganism, and optionally subjecting the microorganism to one or more pre-treatment step(s),
b) lysing the microorganism thereby preparing a lysate comprising an aqueous liquid fraction comprising nucleic acid and solved native protein of the microorganism, further comprising a step b1) of clearing the lysate, preferably by centrifugation or filtration,
c) separating the nucleic acid from the aqueous liquid fraction comprising anion exchange chromatography and/or anion mixed-mode chromatography comprising:
i) adding a nucleic acid adsorbent immobilized to a solid support, preferably a free-floating solid support, to the aqueous liquid fraction,
ii) optionally stirring or shaking, and
iii) separating the nucleic acid bound to the nucleic acid adsorbent immobilized to the solid support, preferably by sedimentation and optionally filtration,
thereby obtaining an aqueous liquid fraction wherein the aqueous liquid fraction is a nucleic acid reduced aqueous liquid fraction,
d) filtrating the aqueous liquid fraction thereby obtaining a solution comprising purified solved native protein of the microorganism and an aqueous solvent, preferably water or a saline solution,
e) optionally removing at least a part of the aqueous solvent,
f) optionally sterilizing the solution thereby obtaining a sterilized solution comprising purified solved native protein of the microorganism and an aqueous solvent, and
g) optionally removing at least a part of the aqueous solvent of the sterilized solution of step f),
wherein preferably the method further comprises a step of separating lipid from the aqueous liquid fraction.
